(19) United States
(12) Patent Application Publication
(10) Pub. No.: US 2026/0194553 A1
(43) Pub. Date: Jul. 9, 2026

(54) FLUID SYSTEM, BIOCHEMICAL ANALYSIS AND DETECTION PLATFORM AND FLUID OPERATION METHOD

(71) Applicant: MGI TECH CO., LTD., Shenzhen, Guangdong (CN)

(72) Inventors: Jian Lu, Shenzhen (CN); Chutian Xing, Shenzhen (CN); Zihua Niu, Shenzhen (CN); Hao Lu, Shenzhen (CN)

(21) Appl. No.: 19/132,641

(22) PCT Filed: Nov. 29, 2022
(86) PCT No.: PCT/CN2022/134945

§ 371 Date: May 23, 2025

(51) Int. Cl. G01N 35/10; G01N 35/04

(52) U.S. Cl. CPC G01N 35/1097; G01N 35/1002; G01N 2035/0468; G01N 2035/0472; G01N 2035/1027

(57) ABSTRACT

A fluid system, a biochemical analysis and detection platform and a fluid operation method are provided. The fluid system includes: one or more first main flow paths configured to be connected with a reagent storage chamber; a second main flow path; a reaction flow path including a flow cell; a bypass flow path connected in parallel with the reaction flow path; a third main flow path; one or more branched flow paths; a first switching component connected to the one or more first main flow paths, the second main flow path and at least one of the one or more branched flow paths; a second switching component connected to the second main flow path, the reaction flow path and the bypass flow path; and a third switching component connected to the third main flow path, the reaction flow path and the bypass flow path.

### CROSS-REFERENCES TO RELATED APPLICATIONS

This application is a national phase application of PCT Application No. PCT/CN2022/134945, filed Nov. 29, 2022, the entire content of which is incorporated herein by reference for all purposes.

### TECHNICAL FIELD

The embodiments of the present disclosure relate to the technical field of fluid systems, in particular to fluid systems, biochemical analysis and detection platforms and fluid operation methods.

### BACKGROUND ART

Molecular biological detection techniques such as gene sequencing, polymerase chain reaction (PCR), biochips and so on are increasingly being applied in numerous scenarios, such as non-invasive prenatal detection and the detection of infectious disease pathogen like the novel coronavirus. These applications are typically carried out on complex biochemical analysis and detection platforms.

Biochemical analysis and detection platforms generally involve one or more target chambers, where samples and various reactants are transported in a solution state to the target chambers for reaction or detection. Taking a gene sequencer as an example, the DNA sample to be detected is usually immobilized on the surface of a flow cell with internal flow channels. The detection of the DNA sample is a periodic cyclic process, with each cycle achieving the detection of one base. Within a single cycle, a variety of functional reagents or buffer solutions are sequentially transported from storage containers to the flow cell through a fluid system. Other biochemical analysis and detection platforms also have similar fluid systems and liquid transportation processes.

The functional reagents consumed in the above-mentioned processes are often extremely expensive, which is one of the main factors restricting the further popularization and large-scale routine application of molecular biological detection technologies such as gene sequencing. Therefore, reducing reagent costs has always been a key task in the research and development of biochemical analysis and detection platforms. There are mainly two ways to reduce costs: one is to reduce the unit-volume price of the reagent, and the other is to reduce the consumption of the reagent. The unit-volume cost of the reagent is not only strongly related to factors such as formulation and process, but also constrained by supply-demand relationship in the market, so reducing the volume consumption of the reagent is a more effective way to reduce costs. Shortening the length of reagent transportation conduits can intuitively reduce losses, but in many cases, there is a lower limit to the length due to the structural layout of the whole machine. In addition, reducing the diameter of the transportation conduits may lead to an excessive pressure drop in the entire fluid system. In a word, although there is some room for optimization of the physical internal volume of the conduits, it is easy to encounter bottlenecks.

Another strategy to reduce consumption of the reagent is to recover and reuse the reagent. Still taking the gene sequencer as an example, assuming that in the case of no recovery, the reagent with a volume of V1 is used in each cycle, and after the cycle ends, this part of the reagent is no longer used and is discharged as waste liquid. In the case of recovery being performed, the reagent with the volume of V1 is transferred from the storage container to the flow cell for reaction in each cycle, and after the reaction, a volume of V2 of the reagent is recovered, so the net consumption of the reagent is V1-V2, leading to a reduction in the reagent cost.

In the related art, the means of directly reversely-driving the reagent to flow backwards with a power source is generally used to recover part of the reagents for reuse. During the development of the present disclosure, the inventors found the following problems in the above-mentioned related art:

In the actual flow, especially in the technical field of microfluidic control, as the flow velocity is low near the wall surface and high away from the wall surface, the interface between two consecutive reagents is the parabola P shown in FIG. 1. Therefore, the target reagent recovered each time has actually been mixed with other reagents. The reagent backflow means of directly reversely-driving part of the reagent to flow backwards with a power source may cause cross-contamination between the reagents. In some scenarios, such cross-mixing may lead to adverse mutual reactions in addition to reducing the purity of the target reagent. In cases of high recovery rates, other reagents may even enter the initial storage area of the target reagent, resulting in more severe cross-contamination.

### CONTENT OF THE INVENTION

An object of the present disclosure is to provide a fluid system, a biochemical analysis and detection platform, and a fluid operation method, for the purpose of reducing cross-contamination between different reagents during recovery of the reagents and reducing the overall working time of the system.

In a first embodiment of the present disclosure, a fluid system is provided, including:
one or more first main flow paths configured to be connected with at least one reagent storage chamber;
a second main flow path;
a reaction flow path including a flow cell;
a bypass flow path connected in parallel with the reaction flow path;
a third main flow path;
one or more branched flow paths; and
a plurality of switching components, the plurality of switching components including:
a first switching component, which is connected to the one or more first main flow paths, the second main flow path and at least one of the one or more branched flow paths, and is configured such that the second main flow path is selectively communicated with any one of the one or more first main flow paths and the at least one of the one or more branched flow paths, while the remaining flow paths connected with the first switching component are disconnected;
a second switching component, which is connected to the second main flow path, the reaction flow path and the bypass flow path, and is configured such that the second main flow path is selectively communicated with either one of the reaction flow path and the bypass flow path, while the remaining flow paths connected with the second switching component are disconnected; and
a third switching component, which is connected to the third main flow path, the reaction flow path and the bypass flow path, and is configured such that the third main flow path is selectively communicated with either one of the reaction flow path and the bypass flow path, while the remaining flow paths connected with the third switching component are disconnected.

In the fluid system in some embodiments, the reaction flow path further includes:
a first flow cell flow path connecting the flow cell with the second switching component; and/or
a second flow cell flow path connecting the flow cell with the third switching component.

In the fluid system in some embodiments, the reaction flow path includes two or more flow cells connected in parallel.

In the fluid system in some embodiments, the one or more branched flow paths include a first waste-liquid flow path, which is connected with the first switching component and configured to be connected with a waste-liquid storage chamber.

In the fluid system in some embodiments,
the fluid system further includes one or more fourth main flow paths, which are configured to be connected with at least one reagent storage chamber and/or at least one waste-liquid storage chamber;
the plurality of switching components further include a fourth switching component, which is connected to the third main flow path, the one or more fourth main flow paths and at least one of the one or more branched flow paths, and configured such that the third main flow path is selectively communicated with any one of the one or more fourth main flow paths and the at least one of the one or more branched flow paths, while the remaining flow paths connected with the fourth switching component are disconnected.

In the fluid system in some embodiments, the one or more branched flow paths include a second waste-liquid flow path, which is connected with the fourth switching component and configured to be connected with a waste-liquid storage chamber.

In the fluid system in some embodiments, the one or more branched flow paths include a storage flow path, which includes:
a storage cell;
a first storage cell connection flow path connecting the first switching component with the storage cell; and
a second storage cell connection flow path connecting the fourth switching component with the storage cell.

In the fluid system in some embodiments, the storage flow path further includes a storage cell inlet flow path connected to the storage cell and/or a storage cell outlet flow path connected to the storage cell.

In the fluid system in some embodiments, the one or more branched flow paths include a plurality of the storage flow paths arranged in parallel, and the plurality of the storage flow paths are selectively connected with the first switching component and/or the fourth switching component.

In the fluid system in some embodiments, at least one of the plurality of switching components is a rotary valve or a solenoid valve.

In the fluid system in some embodiments, the fluid system includes a driving mechanism for driving a fluid within the fluid system to flow, wherein the driving mechanism drives the fluid to flow by positive pressure and/or negative pressure.

In a second aspect of the present disclosure, a biochemical analysis and detection platform is provided, which includes the fluid system described in the first aspect of the present disclosure.

In some embodiments, the biochemical analysis and detection platform includes a molecular biological detection device, and the molecular biological detection device includes the fluid system.

In the biochemical analysis and detection platform in some embodiments, the molecular biological detection device includes a gene sequencer, which includes the fluid system.

In a third aspect of the present disclosure, a fluid operation method of the fluid system described in the first aspect of the present disclosure is provided. The fluid operation method includes:
allowing a reagent to enter at least an end of the bypass flow path close to the second switching component as well as the reaction flow path through the second main flow path;
disconnecting the reaction flow path from the second main flow path and the third main flow path, the reagent being undergoing biochemical reactions within the flow cell of the reaction flow path; and
recovering the reagent in the bypass flow path and/or the reaction flow path.

In the fluid operation method in some embodiments, while the reagent is undergoing biochemical reactions within the flow cell of the reaction flow path, the reagent in the bypass flow path is recovered.

In the fluid operation method in some embodiments, part of the reagent in the bypass flow path is made to flow to the second main flow path so as to recover the reagent in the bypass flow path through the second main flow path.

In the fluid operation method in some embodiments, the reagent in the reaction flow path is made to flow to the second main flow path so as to recover the reagent in the reaction flow path through the second main flow path.

In the fluid operation method in some embodiments, the fluid operation method further includes allowing the reagent, recovered through the second main flow path, to flow to the first main flow path.

In the fluid operation method in some embodiments, the fluid operation method further includes allowing the reagents, recovered from the bypass flow path and the reaction flow path through the second main flow path, to flow to the third main flow path to recover the reagent through the third main flow path.

In the fluid operation method in some embodiments, the fluid system further includes a storage flow path connected with the first switching component and a fourth switching component. The fluid operation method includes allowing the reagent, recovered through the third main flow path, to flow to the storage flow path.

In the fluid operation method in some embodiments, the fluid operation method includes pushing the reagent to flow within the fluid system by a buffer solution to recover the reagent.

In the fluid operation method in some embodiments, the fluid operation method includes discharging the buffer solution or a mixture of the buffer solution and the reagent whose concentration is less than a predetermined concentration into a waste-liquid storage chamber.

In the fluid operation method in some embodiments,
the fluid operation method includes discharging the buffer solution or a mixture of the buffer solution and the reagent whose concentration is less than a predetermined concentration into the waste-liquid storage chamber through at least one of the one or more branched flow paths; and/or
the fluid system includes a fourth main flow path, which is selectively communicated with the third main flow path and configured to be connected to the waste-liquid storage chamber. The fluid operation method includes discharging the buffer solution or a mixture of the buffer solution and the reagent whose concentration is less than a predetermined concentration into the waste-liquid storage chamber through the fourth main flow path.

Based on the fluid system provided in the present disclosure, by introducing a bypass flow path at the upstream end of the flow cell, the reagent with a lower concentration is led away through the bypass flow path every time before the reagent enters the flow cell for reaction. This helps to ensure that the reagent entering the flow cell has a sufficiently high concentration. If different reagents are not allowed to come into contact with each other, a buffer solution may be introduced with the participation of the bypass flow path and the branched flow paths to isolate the different reagents from each other, which is beneficial to reducing cross-contamination between the reagents. The fluid system provided in the present disclosure can also have the bubbles, which are not allowed to enter the flow cell, discharged through the bypass flow path before the reagent enters the flow cell for reaction. Additionally, the fluid system provided in the present disclosure can enable concurrent operations of sample reaction and reagent recovery, and thus can shorten the waiting time of relevant steps and improve the overall working efficiency.

The biochemical analysis and detection platform and the fluid operation method provided in the present disclosure possess the advantages of the fluid system provided in the present disclosure.

Other features and advantages of the present disclosure will become clear through the following detailed description of exemplary embodiments of the present disclosure with reference to the attached drawings.

### BRIEF DESCRIPTION OF THE DRAWINGS

The accompanying drawings described herein are provided for further understanding of the embodiments of the present disclosure and constitute part of the present disclosure. The illustrative embodiments of the present disclosure and their descriptions are used to explain the embodiments of the present disclosure and do not constitute undue limitations on the embodiments of the present disclosure. In the figures,

FIG. 1 is a schematic view showing the shape of an interface between reagents flowing in a flow channel.

FIG. 2 is a schematic principled view of a fluid system according to an embodiment of the present disclosure.

FIGS. 3 to 5 are schematic principled views of the related fluid operation methods of the fluid system in the embodiment shown in FIG. 2.

FIG. 6 is a schematic principled view of a fluid system according to an embodiment of the present disclosure.

FIGS. 7 to 10 are schematic principled views of the related fluid operation methods of the fluid system in the embodiment shown in FIG. 6.

FIG. 11 is a schematic principled view of a fluid system according to an embodiment of the present disclosure.

FIG. 12 is a schematic principled structural view of a fluid system according to an embodiment of the present disclosure.

FIGS. 13 and 14 are schematic principled structural views of the related fluid operation methods of the fluid system in the embodiment shown in FIG. 12.

FIG. 15 is a schematic view showing the distribution of reagents when the recovered reagent, the mixed reagent and a fresh reagent coexist in a conduit in the embodiment shown in FIG. 12.

FIG. 16 is a schematic principled structural view of a fluid system according to an embodiment of the present disclosure.

FIGS. 17 and 18 are schematic principled structural views of the related fluid operation methods of the fluid system in the embodiment shown in FIG. 16.

FIG. 19 is a schematic principled structural view of a fluid system according to an embodiment of the present disclosure.

FIG. 20 is a partial schematic structural view of the fluid system of FIG. 19.

### EMBODIMENTS

The technical solutions in the embodiments of the present disclosure will be clearly and completely described with reference to the drawings in the embodiments of the present disclosure. Obviously, the described embodiments are only part of the embodiments of the present disclosure other than the whole embodiments. The following description for at least one exemplary embodiment is merely illustrative in actual and is in no way intended to limit the embodiments of the present disclosure and its application or uses. All other embodiments that are obtained by those skilled in the art based on the embodiments of the present disclosure without paying inventive effort fall within the protection scope of the embodiments of the present disclosure.

Unless otherwise specified, the relative arrangement of components and steps, numerical expressions and numerical values set forth in these embodiments do not limit the scope of the embodiments of the present disclosure. It should be understood that the dimensions of various parts shown in the drawings are not drawn to actual scale for the sake of convenience in description. Techniques, methods and equipment known to those skilled in the art in the related art may not be discussed in detail, but they should be regarded as part of the description under appropriate circumstances. In all examples shown and discussed herein, any specific values should be interpreted as illustrative only and not as a limitation. Therefore, other examples of the exemplary embodiments may have different values. It should be noted that similar reference numerals and letters denote similar items in the following drawings. Thus, once an item is defined in one drawing, it does not need to be further discussed in subsequent drawings.

In an embodiment of the present disclosure, a fluid system is provided, which includes one or more first main flow paths L6, a second main flow path L1, a reaction flow path, a bypass flow path L5, one or more branched flow paths, and a plurality of switching components.

The first main flow path L6 is configured to be connected with at least one reagent storage chamber.

The reaction flow path includes a flow cell C1.

The bypass flow path L5 is connected in parallel with the reaction flow path.

The plurality of switching components include a first switching component T1, a second switching component T2, and a third switching component T3.

The first switching component T1 is connected to the one or more first main flow paths L6, the second main flow path L1 and at least one of the one or more branched flow paths, and is configured to selectively communicate the second main flow path L1 with any one of the one or more first main flow paths L6 and the at least one of the one or more branched flow paths while having the remaining flow paths connected with the first switching component T1 disconnected.

The second switching component T2 is connected to the second main flow path L1, the reaction flow path and the bypass flow path L5, and is configured to selectively communicate the second main flow path L1 with either one of the reaction flow path and the bypass flow path L5 while having the remaining flow paths connected with the second switching component T2 disconnected.

The third switching component T3 is connected to the third main flow path L4, the reaction flow path and the bypass flow path L5, and is configured to selectively communicate the third main flow path L4 with either one of the reaction flow path and the bypass flow path L5 while having the remaining flow paths connected with the third switching component T3 disconnected.

In the fluid system in embodiments of the present disclosure, by introducing the bypass flow path L5 at the upstream end of the flow cell C1, the reagent with lower concentration or bubbles can be diverted through the bypass flow path L5 every time before the reagent enters the flow cell C1 for reaction. This helps to ensure that the reagent entering the flow cell C1 has a sufficiently high concentration. If different reagents are not allowed to come into contact with each other, a buffer solution may be introduced to isolate different reagents with the participation of the bypass flow path and the branched flow path, which is beneficial to reduce cross-contamination between reagents. Additionally, the bubbles that are not allowed to enter the flow cell C1 can be discharged through the bypass flow path L5.

The fluid system provided in the present disclosure can also have the bubbles, which are not allowed to enter the flow cell C1, discharged through the bypass flow path L5 before the reagent enters the flow cell for reaction.

Further, the introduction of the bypass flow path L5 and the addition of the branched flow paths enables concurrent operation of the reagent recovery and the biochemical reactions, which can shorten the waiting time of relevant steps and improve the overall working efficiency.

In the fluid system in some embodiments, the reaction flow path further includes a first flow cell flow path L2 and/or a second flow cell flow path L3. The first flow cell flow path L2 connects the flow cell C1 with the second switching component T2. The second flow cell flow path L3 connects the flow cell C1 with the third switching component T3.

In the fluid system in some embodiments, the reaction flow path includes two or more flow cells C1 arranged in parallel.

In the fluid system in some embodiments, the one or more branched flow paths include a first waste-liquid flow path L11, which is connected with the first switching component T1 and configured to be connected to a waste-liquid storage chamber.

In the fluid system in some embodiments, the fluid system further includes one or more fourth main flow paths L7, which are configured to be connected to at least one reagent storage chamber and/or at least one waste-liquid storage chamber; the plurality of switching components further include a fourth switching component T4, which is connected to the third main flow path L4, the one or more fourth main flow paths L7 and at least one of the branched flow paths, and is configured to selectively communicate the third main flow path L4 with any one of the one or more fourth main flow paths L7 and the at least one branched flow path while having the remaining flow paths connected with the fourth switching component T4 are disconnected.

Regarding the types of the flow paths, the first to fourth main flow paths belong to different types. For example, when a plurality of the first main flow paths L6 are included, all the first main flow paths L6 are of the same type; when a plurality of the fourth main flow paths L7 are included, all the fourth main flow paths L7 are of the same type; when a plurality of the branched flow paths are included, all the branched flow paths are of the same type. For another example, any two of the first main flow path L6, the second main flow path L1 and the branched flow path belong to different types of the flow paths, and any two of the fourth main flow path L7, the second main flow path and the branched flow path belong to different types of the flow paths.

In the fluid system in some embodiments, the one or more branched flow paths include a second waste-liquid flow path L12, which is connected with the fourth switching component T4 and configured to be connected to a waste-liquid storage chamber.

In the fluid system in some embodiments, the one or more branched flow paths include a storage flow path, which includes a storage cell C2, a first storage cell connection flow path L8 and a second storage cell connection flow path L9. The first storage cell connection flow path L8 connects the first switching component T1 with the storage cell C2. The second storage cell connection flow path L9 connects the fourth switching component T4 with the storage cell C2.

In the fluid system in some embodiments, the storage flow path further includes a storage cell inlet flow path L10 connected to the storage cell C2 and/or a storage cell outlet flow path L13 connected to the storage cell C2.

In the fluid system in some embodiments, the one or more branched flow paths include a plurality of the storage flow paths arranged in parallel.

In the fluid system in some embodiments, at least one of the plurality of switching components is a rotary valve or a solenoid valve.

In the fluid system in some embodiments, the fluid system includes a driving mechanism for driving the fluid within the fluid system to flow, wherein the driving mechanism drives flow of the fluid by means of positive pressure and/or negative pressure.

Also provided in the embodiments of the present disclosure is a biochemical analysis and detection platform, which includes the fluid system described in the embodiments of the present disclosure. The biochemical analysis and detection platform provided in the embodiments of the present disclosure has the same advantages as the fluid system described in the embodiments of the present disclosure.

The biochemical analysis and detection platform includes, for example, a molecular biological detection device, which includes the fluid system according to embodiments of the present disclosure. As the molecular biological detection device includes the fluid system described in the embodiments of the present disclosure, it can also adopt the fluid operation method according to embodiments of the present disclosure.

The molecular biological detection device includes, for example, a gene sequencer, which includes the fluid system according to embodiments of the present disclosure. As the gene sequencer includes the fluid system described in the embodiments of the present disclosure, it can also adopt the fluid operation method according to embodiments of the present disclosure.

In the embodiments of the present disclosure, a fluid operation method for the fluid system described in the embodiments of the present disclosure is further provided, which includes: allowing a reagent to enter the reaction flow path and at least an end of the bypass flow path L5 close to the second switching component T2 through the second main flow path L1; disconnecting the reaction flow path from the second main flow path L1 and the third main flow path L4 to allow the reagent to undergo biochemical reactions within the flow cell C1 of the reaction flow path; and recovering the reagent in the bypass flow path L5 and/or the reaction flow path.

The fluid operation method provided in the embodiments of the present disclosure has the same advantages as the fluid system provided in the embodiments of the present disclosure.

In the fluid operation method in some embodiments, while the reagent is undergoing biochemical reactions within the flow cell C1 of the reaction flow path, the reagent in the bypass flow path L5 is recovered.

In the fluid operation method in some embodiments, part of the reagent in the bypass flow path L5 is made to flow to the second main flow path L1 to recover the reagent in the bypass flow path L5 by the second main flow path L1.

In the fluid operation method in some embodiments, the reagent in the reaction flow path is made to flow to the second main flow path L1 to recover the reagent in the reaction flow path by the second main flow path L1.

In the fluid operation method in some embodiments, the fluid operation method further includes allowing the reagent recovered by the second main flow path L1 to flow to the first main flow path L6.

In the fluid operation method in some embodiments, the fluid operation method further includes allowing the reagent, recovered from the bypass flow path L5 and the reaction flow path by the second main flow path L1, to flow to the third main flow path L4 to recover the reagent by the third main flow path L4.

In the fluid operation method in some embodiments, the fluid system further includes a storage flow path connected to the first switching component T1 and the fourth switching component T4. The fluid operation method includes allowing the reagent, which is recovered by the third main flow path L4, to flow to the storage flow path.

In the fluid operation method in some embodiments, the fluid operation method includes pushing the reagent to flow within the fluid system by a buffer solution to recover the reagent.

In the fluid operation method in some embodiments, the fluid operation method includes discharging the buffer solution or a mixture of the buffer solution and the reagent whose concentration is less than a predetermined concentration into the waste-liquid storage chamber.

In the fluid operation method in some embodiments, the fluid operation method includes discharging the buffer solution or a mixture of the buffer solution and the reagent whose concentration is less than a predetermined concentration into the waste-liquid storage chamber through at least one of the one or more branched flow paths; and/or the fluid system includes a fourth main flow path L7, which is selectively communicated with the third main flow path L4 and configured to be connected to the waste-liquid storage chamber. The fluid operation method includes discharging the buffer solution or a mixture of the buffer solution and the reagent whose concentration is less than a predetermined concentration into the waste-liquid storage chamber through the fourth main flow path L7.

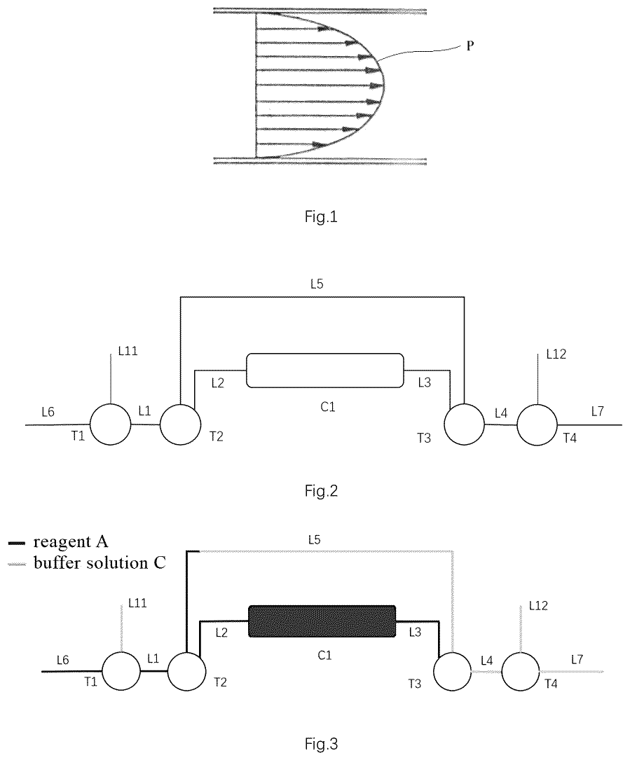

The fluid system and the fluid operation method according to the embodiments of the present disclosure will be described in more detail with reference to FIGS. 2 to 20.

FIGS. 2 to 5 are schematic principled views according to an embodiment of the present disclosure. FIG. 2 is a schematic principled view of the fluid system in this embodiment. FIGS. 3 to 5 are schematic principled views of the related fluid operation methods of the fluid system in the embodiment shown in FIG. 2.

As shown in FIGS. 2 to 5, the fluid system in this embodiment of the present disclosure includes a first main flow path L6, a second main flow path L1, a reaction flow path, a bypass flow path L5, a third main flow path L4, a fourth main flow path L7, a first waste-liquid flow path L11 as a branched flow path, a second waste-liquid flow path L12 as a branched flow path, a first switching component T1, a second switching component T2, a third switching component T3, and a fourth switching component T4.

The reaction flow path includes a flow cell C1, and a first flow cell flow path L2 and a second flow cell flow path L3 connected to the flow cell C1 respectively. The bypass flow path L5 is connected in parallel with the reaction flow path. The first waste-liquid flow path L11 is configured to be connected to the waste-liquid storage chamber. The second waste-liquid flow path L12 is configured to be connected to the waste-liquid storage chamber. The first switching component T1 is connected to the first main flow path L6, the second main flow path L1 and the first waste-liquid flow path L11, and is configured to selectively communicate any two of the first main flow path L6, the second main flow path L1 and the first waste-liquid flow path L11 while having the remaining flow paths disconnected from the two communicated flow paths. The second switching component T2 is connected to the second main flow path L1, the first flow cell flow path L2 and the bypass flow path L5, and is configured to selectively communicate any two of the second main flow path L1, the first flow cell flow path L2 and the bypass flow path L5 while having the remaining flow paths disconnected from the two communicated flow paths. The third switching component T3 is connected to the third main flow path L4, the second flow cell flow path L3 and the bypass flow path L5, and is configured to selectively communicate any two of the third main flow path L4, the second flow cell flow path L3 and the bypass flow path L5 while having the remaining flow paths disconnected from the two communicated flow paths. The fourth switching component T4 is connected to the third main flow path L4, the fourth main flow path L7 and the second waste-liquid flow path L12, and is configured to selectively communicate any two of the third main flow path L4, the fourth main flow path L7 and the second waste-liquid flow path L12 while having the remaining flow paths disconnected from the two communicated flow paths.

The logic timing solution of a feasible fluid operation method including a reagent recovery process according to embodiments of the present disclosure will be described with reference to FIGS. 2 to 5. In this logical timing solution, a reagent A is the reagent that needs to be partially recovered, a reagent B is the reagent that cannot cross-contaminate with the reagent A and does not need to be recovered, and a buffer solution C serves as an intermediate medium to separate the reagent A from the reagent B. These three liquids, namely the reagent A, the reagent B, and the buffer solution C, constitute the minimal component system of the reagent recovery solution. In practical applications, the component system of the reagent recovery solution can be expanded. For example, the reagents to be recovered may include various reagents, such as A1, A2, A3 . . . . An.

In the logical timing solution of the embodiments of the present disclosure, the first main flow path L6 is described as the upstream of the fluid system. The upstream of the first main flow path L6 is in fluid communication with a storage chamber of the reagent A; the downstream of the fourth main flow path L7 is in fluid communication with storage chambers of the other reagents (such as the reagent B, the buffer solution C, etc.); the downstream of the first waste-liquid flow path L11 and the second waste-liquid flow path L12 is in fluid communication with the waste-liquid storage chambers.

The logical timing solution of the fluid operation method involves the following steps:

Step S1100: In the initial state, the first main flow path L6 contains the reagent A; the flow cell C1 contains the buffer solution C, and the second main flow path L1, the first flow cell flow path L2, the second flow cell flow path L3, the third main flow path L4, the bypass flow path L5 and the fourth main flow path L7 all contain the buffer solution C; the reagent B has not yet entered the fluid system. The first switching component T1 communicates the first main flow path L6 with the second main flow path L1; the second switching component T2 communicates the second main flow path L1 with the bypass flow path L5; the third switching component T3 communicates the bypass flow path L5 with the third main flow path L4; and the fourth switching component T4 communicates the third main flow path L4 with the second waste-liquid flow path L12.

Step S1101: The reagent A enters the second main flow path L1 from the first main flow path L6 through the first switching component T1 and then enters the bypass flow path L5 through the second switching component T2 to replace the buffer solution C in the second main flow path L1 and the bypass flow path L5, until the first main flow path L6 and the second main flow path L1 are filled up with the reagent A with a concentration greater than 99%, and the bypass flow path L5 contains a mixture of the reagent A and the buffer solution C. The excess buffer solution C flows sequentially through the third switching component T3, the third main flow path L4, the fourth switching component T4 and the second waste-liquid flow path L12 before being discharged. This step S1101 is to ensure the high concentration of the reagent A entering the flow cell C1.

Step S1102: The second switching component T2 is switched to bring the second main flow path L1 into fluid communication with the first flow cell flow path L2; the third switching component T3 is switched to bring the second flow cell flow path L3 into fluid communication with the third main flow path L4. At this time, both the upstream of the second switching component T2 and the downstream of the third switching component T3 are in fluid communication with the flow cell C1. The reagent A with high concentration enters the flow cell C1 through the second switching component T2 and the first flow cell flow path L2 to replace the buffer solution in the first flow cell flow path L2, the flow cell C1 and the second flow cell flow path L3, as shown in FIG. 3, until the first flow cell flow path L2, the flow cell C1 and the second flow cell flow path L3 are filled up with the reagent A with a concentration greater than 99%, and the third main flow path L4 contains a mixture of the reagent A and the buffer solution C.

Step S1103: The DNA sample fixed inside the flow cell C1 immediately undergoes a biochemical reaction with the reagent A. Concurrently, the second switching component T2 is switched to bring the second main flow path L1 into fluid communication with the bypass flow path L5 while the upstream of the second switching component T2 is disconnected from the flow cell C1; the third switching component T3 is switched to bring the third main flow path L4 into fluid communication with the bypass flow path L5 while the downstream of the third switching component T3 is disconnected from the flow cell C1; the fourth switching component T4 is switched to bring the third main flow path L4 into fluid communication with the fourth main flow path L7.

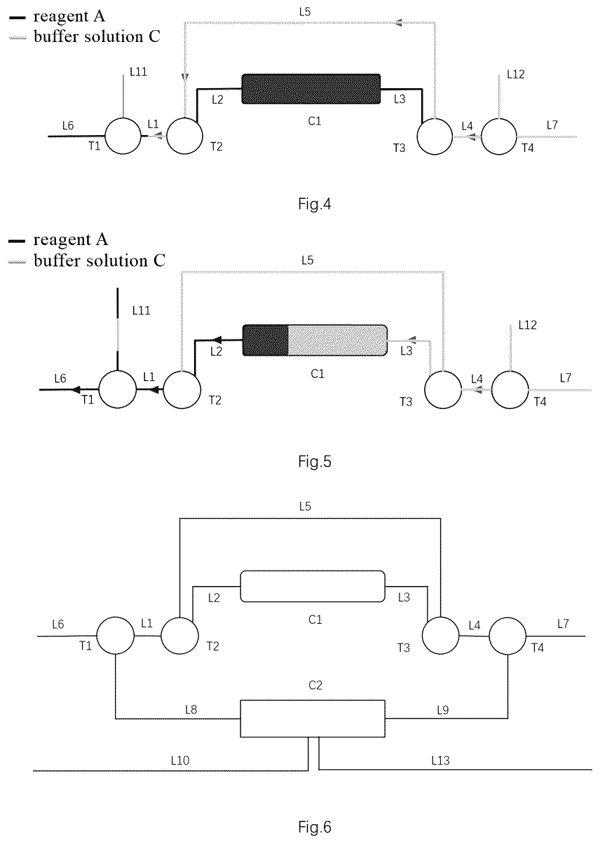

Step S1104: The buffer solution C enters the third main flow path L4 from the fourth main flow path L7 through the fourth switching component T4, and then enters the bypass flow path L5 through the third switching component T3 to replace the reagent A with high concentration at the end of the bypass flow path L5 close to the second switching component T2 and that in the second main flow path L1 and the first main flow path L6. All of the reagent A with a concentration greater than 95% is sent back to the first main flow path L6 until the second main flow path L1 contains a mixture of the reagent A and the buffer solution C, and the bypass flow path L5 contains the buffer solution C, as shown in FIG. 4.

Step S1105: The first switching component T1 is switched to bring the second main flow path L1 into fluid communication with the first waste-liquid flow path L11. After step S1104, the buffer solution C continues to enter the fluid system, and the buffer solution C is continuously filled into the third main flow path L4 and the bypass flow path L5 until the fourth main flow path L7, the third main flow path L4 and the bypass flow path L5 are filled up with the buffer solution C with a concentration greater than 99%. The excess buffer solution C and the reagent A with low concentration flow sequentially through the second switching component T2, the second main flow path L1, the first switching component T1, and the first waste-liquid flow path L11 before being discharged. Steps S1104 and S1105 can be performed concurrently with the biochemical reaction occurring within the flow cell C1 and they take less time than the biochemical reaction within the flow cell C1.

Step S1106: After the biochemical reaction within the flow cell C1 is completed, the third switching component T3 is switched to bring the second flow cell flow path L3 into fluid communication with the third main flow path L4, thus establishing a communication between the flow cell C1 and the reagent storage chamber downstream of the fourth main flow path L7; the second switching component T2 is switched to bring the second main flow path L1 into fluid communication with the first flow cell flow path L2, thus establishing a fluid communication between the flow cell C1 and the waste-liquid storage chamber through the first waste-liquid flow path L11. The buffer solution C with high concentration enters the flow cell C1 through the third switching component T3 and the second flow cell flow path L3 to replace the reagent A in the second flow cell flow path L3, the flow cell C1 and the first flow cell flow path L2 as well as the mixture in the second main flow path L1, and the mixture and the reagent A with low concentration are discharged through the first waste-liquid flow path L11, until the second main flow path L1, the first flow cell flow path L2, and the side of the flow cell C1 close to the second switching component T2 are filled up with the reagent A with high concentration.

Step S1107: The first switching component T1 is switched to bring the second main flow path L1 into fluid communication with the first main flow path L6, and the buffer solution C continues to enter the flow cell C1. The reagent A with a concentration greater than 95% in the first flow cell flow path L2, the flow cell C1 and the second flow cell flow path L3 enters the first main flow path L6, as shown in FIG. 5. So far, the recovery process of the reagent A is completed.

Step S1108: the first switching component T1 is switched to bring the second main flow path L1 into fluid communication with the first waste-liquid flow path L11. Subsequently, the reagent A with low concentration and the mixture of the reagent A and the buffer solution C in the second main flow path L1, the first flow cell flow path L2 and the flow cell C1 are discharged through the first waste-liquid flow path L11, until the second main flow path L1, the first flow cell flow path L2, the second flow cell flow path L3, the third main flow path L4, the bypass flow path L5, the fourth main flow path L7 and the flow cell C1 are all filled up with the buffer solution C.

Step S1109: The second switching component T2 is switched to establish fluid communication between the second main flow path L1 and the bypass flow path L5. The switching component T3 is switched to establish fluid communication between the third main flow path L4 and the bypass flow path L5. The flow cell C1 is disconnected from both the upstream of the second switching component T2 and the downstream of the third switching component T3, thereby being disconnected from the reagent storage chambers upstream of the first main flow path L6 and downstream of the fourth main flow path L7. The reagent B enters from the fourth main flow path L7, and passes through the fourth switching component T4 and the third main flow path L4 into the bypass flow path L5 to replace the buffer solution C in the third main flow path L4 and the bypass flow path L5, until the fourth main flow path L7 and the third main flow path L4 are filled up with the reagent B with a concentration greater than 99%, and the bypass flow path L5 contains a mixture of the reagent B and the buffer solution C. The excess buffer solution C in the bypass flow path L5 flows sequentially through the second switching component T2, the second main flow path L1, the first switching component T1 and the first waste-liquid flow path L11 before being discharged. Step S1109 is to ensure the high concentration of the reagent B entering the flow cell C1.

Step S1110: the second switching component T2 is switched to bring the second main flow path L1 into fluid communication with the first flow cell flow path L2; the third switching component T3 is switched to bring the second flow cell flow path L3 into fluid communication with the third main flow path L4. At this time, both the upstream of the second switching component T2 and the downstream of the third switching component T3 are in fluid communication with the flow cell C1. The reagent B with high concentration enters the flow cell C1 through the third switching component T3 and the second flow cell flow path L3 to replace the buffer solution C in the second flow cell flow path L3, the flow cell C1 and the first flow cell flow path L2, until the second flow cell flow path L3, the flow cell C1 and the first flow cell flow path L2 are filled up with the reagent B with a concentration greater than 99%, and the second main flow path L1 and the first waste-liquid flow path L11 contain a mixture of the reagent B and the buffer solution C.

Step S1111: The DNA sample fixed inside the flow cell C1 immediately undergoes a biochemical reaction with the reagent B. Concurrently, the second switching component T2 is switched to bring the second main flow path L1 into fluid communication with the bypass flow path L5 while the upstream of the second switching component T2 is disconnected from the flow cell C1; the third switching component T3 is switched to bring the third main flow path L4 into fluid communication with the bypass flow path L5 while the downstream of the third switching component T3 is disconnected from the flow cell C1.

Step S1112: The buffer solution C enters the third main flow path L4 from the fourth main flow path L7 through the fourth switching component T4, and then enters the bypass flow path L5 through the third switching component T3 to replace the reagent B in the bypass flow path L5 and the second main flow path L1. All of the reagent B is discharged through the first waste-liquid flow path L11 until the second main flow path L1, the bypass flow path L5, the third main flow path L4 and the fourth main flow path L7 are filled up with the buffer solution C.

Step S1113: After the biochemical reaction of the reagent B is completed, the second switching component T2 is switched to bring the second main flow path L1 into fluid communication with the first flow cell flow path L2; the third switching component T3 is switched to bring the second flow cell flow path L3 into fluid communication with the third main flow path L4. At this time, both the upstream of the second switching component T2 and the downstream of the third switching component T3 are in fluid communication with the flow cell C1. The buffer solution C enters the flow cell C1 through the third switching component T3 and the second flow cell flow path L3 to replace the reagent B in the second flow cell flow path L3, the flow cell C1 and the first flow cell flow path L2. So far, all of the flow paths except for the first main flow path L6, the first waste-liquid flow path L11 and the second waste-liquid flow path L12, as well as the flow cell are filled up with the buffer solution C, and the reagent B has been discharged through a recovery flow path.

Repeating Steps S1100 to S1113 may initiate a new cycle. Throughout the fluid operation method, the reagent A and the reagent B are separated by the buffer solution C and may not come into contact, thereby preventing cross-contamination between the reagents.

In the logic timing solution of the fluid operation method described above, the operation performed on the reagent A is to recover part of the reagent A, while the operation performed on the reagent B is not to recover the reagent B. However, according to the logic timing solution described above, the steps for partial recovery of the reagent A may also be applied to recover the reagent B. Additionally, molecular biological detection involving more reagents can be implemented according to the above-mentioned method steps, and whether the corresponding reagents are recovered or not can be selected.

In the logic timing solution of the fluid operation method described above, two reagents are taken as an example, where the reagent B and the buffer solution C both enter from the fourth main flow path L7, while the reagent A enters through the first main flow path L6. In the case where more reagents participate in molecular biological detection, multiple reagent storage chambers may be provided upstream of the first switching component T1 and downstream of the fourth switching component T4 respectively to store different reagents, and these reagent storage chambers storing different reagents may be selectively connected to the first main flow path L6 or the fourth main flow path L7. Alternatively, in an embodiment not illustrated, a plurality of first main flow paths L6 connected to different reagent storage chambers may be directly connected to the first switching component T1, and/or a plurality of fourth main flow paths L7 connected to different reagent storage chambers may be directly connected to the fourth switching component T4, so that different reagents can be introduced into the fluid system according to reaction requirements, and if necessary, the buffer solution C may be used to isolate different reagents to avoid cross-contamination caused by mutual contact of different reagents.

In the embodiments of the present disclosure, when the reagent A, the reagent B, and the buffer solution Center the fluid system, they can be pushed in by positive pressure from the inlet side of the fluid flow direction, for example, using a diaphragm liquid pump or a syringe pump upstream of the first main flow path L6 to push the liquid. Alternatively, they can be sucked in by negative pressure coming from the outlet side of the fluid flow direction, for example, using a syringe pump at end where the fourth main flow path L7/the second waste-liquid flow path L12 is located to suck the reagent at the inlet end of the first main flow path L6.

In the embodiments of the present disclosure, only one reaction flow path is illustrated. However, in the embodiments not shown, multiple reaction flow paths may be arranged in parallel to form a multi-input and multi-output reaction area.

In the logic timing solution of the fluid operation method described above, the reagent A may be recovered to the first main flow path L6 and its upstream, and part of the reagent at the end close to the flow cell C1 is consumed each time. Considering the dilution of concentration in the flow cell C1 and in the flow paths connected with the flow cell C1, the recovery amount is limited. On the premise that the cross-section of the flow cell C1 is a wide and shallow rectangle, if the reagent with a concentration of more than 95% needs to be recovered, the recovery ratio is generally less than 25%.

Considering that each biochemical process does not take long, the concentration of the reagent in the first main flow path L6 cannot reach uniform only by the diffusion effect, wherein the concentration of the reagent close to the second main flow path L1 is relatively low, while away from the second main flow L1 is a fresh reagent. The portion entering the bypass flow path L5 every time and the small portion first entering the flow cell C1 may be the reagent with relatively low concentration, followed by entrance of the fresh reagent. In the liquid pumping process, these two parts of the reagents, when passing through the switching components and the flow paths, may be uniformly mixed to some extent. Therefore, the reagent in the flow cell C1 is relatively uniform in the end, but the concentration thereof is slightly lower than that of the fresh reagent.

In the logic timing solution of the fluid operation method described above, some steps of the reagent recovery process are performed concurrently with the biochemical reaction occurring in the flow cell C1, which is beneficial for saving time for molecular biological detection.

FIGS. 6 to 10 are schematic principled views according to an embodiment of the present disclosure. FIG. 6 is a schematic principled view of a fluid system according to an embodiment of the present disclosure. FIGS. 7 to 10 are schematic principled views of the related fluid operation methods of the fluid system in the embodiment shown in FIG. 6.

As shown in FIGS. 6 to 10, the fluid system in this embodiment of the present disclosure includes a first main flow path L6, a second main flow path L1, a reaction flow path, a bypass flow path L5, a third main flow path L4, a fourth main flow path L7, a storage flow path as a branched flow path, a first switching component T1, a second switching component T2, a third switching component T3, a fourth switching component T4.

The reaction flow path includes a flow cell C1, and a first flow cell flow path L2 and a second flow cell flow path L3 connected to the flow cell C1 respectively. The bypass flow path L5 is connected in parallel with the reaction flow path. The storage flow path includes a storage cell C2, a first storage cell connection flow path L8 and a second storage cell connection flow path L9 connected to the storage cell C2, as well as a storage cell inlet flow path L10 and a storage cell outlet flow path L13 connected to the storage cell C2. The first switching component T1 is connected to the first main flow path L6, the second main flow path L1 and the first storage cell connection flow path L8, and is configured to selectively communicate any two of the first main flow path L6, the second main flow path L1 and the first storage cell connection flow path L8 while having the remaining flow paths disconnected from the two communicated flow paths. The second switching component T2 is connected to the second main flow path L1, the first flow cell flow path L2 and the bypass flow path L5, and is configured to selectively communicate any two of the second main flow path L1, the first flow cell flow path L2 and the bypass flow path L5 while having the remaining flow paths disconnected from the two communicated flow paths. The third switching component T3 is connected to the third main flow path L4, the second flow cell flow path L3 and the bypass flow path L5, and is configured to selectively communicate any two of the third main flow path L4, the second flow cell flow path L3 and the bypass flow path L5 while having the remaining flow paths disconnected from the two communicated flow paths. The fourth switching component T4 is connected to the third main flow path L4, the fourth main flow path L7 and the second storage cell connection flow path L9, and is configured to selectively communicate any two of the third main flow path L4, the fourth main flow path L7 and the second storage cell connection flow path L9 while having the remaining flow paths disconnected from the two communicated flow paths.

The logic timing solution of a feasible fluid operation method including a reagent recovery process according to embodiments of the present disclosure will be described with reference to FIGS. 6 to 10. In this logical timing solution, the reagent A is the reagent that needs to be partially recovered, the reagent B is the reagent that cannot cross-contaminate with the reagent A and does not need to be recovered, and the buffer solution C serves as an intermediate medium to separate the reagent A from the reagent B. These three liquids, namely the reagent A, the reagent B, and the buffer solution C, constitute the minimal component system of the reagent recovery solution. In practical applications, the component system of the reagent recovery solution can be expanded. For example, the reagents to be recovered may include various reagents, namely A1, A2, A3 . . . . An.

In the logical timing solution of the fluid operation method according to embodiments of the present disclosure, the first main flow path L6 is still regarded as the upstream of the fluid system. As shown in FIGS. 6 to 10, in this embodiment, a storage flow path as branched flow path is used in place of the first waste-liquid flow path L11 and the second waste-liquid flow path L12 in the fluid system shown in FIGS. 2 to 5. The storage flow path includes a storage cell C2, a first storage cell connection flow path L8 and a second storage cell connection flow path L9 connected to the storage cell C2, as well as a storage cell inlet flow path L10 and a storage cell outlet flow path L13 connected to the storage cell. At the same time, a one-way liquid pumping means is adopted, so that the reagent enters the flow cell C1 through the first flow cell flow path L2 and flows out of the flow cell C1 through the second flow cell flow path L3; the reagent A enters the storage cell C2 through the storage cell inlet flow path L10 for temporary storage; the upstream of the first main flow path L6 is in fluid communication with the reagent storage chambers of the reagent B and the buffer solution C; the downstream of the fourth main flow path L7 is in fluid communication with the waste-liquid storage chamber.

The logical timing solution of the fluid operation method involves the following steps:

Step S2100: In the initial state, the flow cell C1 contains the buffer solution C; the second main flow path L1, the first flow cell flow path L2, the second flow cell flow path L3, the third main flow path L4, the bypass flow path L5 and the first main flow path L6 all contain the buffer solution C; the first storage cell connection flow path L8 contains the reagent A, while the second storage cell connection flow path L9 contains the air; and the storage cell C2 contains a certain volume of the reagent A. As the amount of the reagent A recovered each time is smaller than the consumption, a certain volume of the reagent A needs to be preloaded in the storage cell C2. The first switching component T1 communicates the first main flow path L6 with the second main flow path L1; the second switching component T2 communicates the second main flow path L1 with the bypass flow path L5; the third switching component T3 communicates the bypass flow path L5 with the third main flow path L4; and the fourth switching component T4 communicates the third main flow path L4 with the fourth main flow path L7.

Step S2101: The first switching component T1 is switched to bring the first storage cell connection flow path L8 into fluid communication with the second main flow path L1. The reagent A enters the second main flow path L1 from the storage cell C2 and the first storage cell connection flow path L8 through the first switching component T1 and then enters the bypass flow path L5 through the second switching component T2 to replace the buffer solution C in the second main flow path L1 and the bypass flow path L5, until the first storage cell connection flow path L8 and the second main flow path L1 are filled up with the reagent A with a concentration greater than 99%, and the bypass flow path L5 contains a mixture of the reagent A and the buffer solution C. The excess buffer solution C flows sequentially through the third switching component T3, the third main flow path L4, the fourth switching component T4 and the fourth main flow path L7 before being discharged. This step S2101 is to ensure the high concentration of the reagent A entering the flow cell C1.

Step S2102: The second switching component T2 is switched to bring the second main flow path L1 into fluid communication with the first flow cell flow path L2; the third switching component T3 is switched to bring the second flow cell flow path L3 into fluid communication with the third main flow path L4. At this time, both the upstream of the second switching component T2 and the downstream of the third switching component T3 are in fluid communication with the flow cell C1. The reagent A with high concentration enters the flow cell C1 through the second switching component T2 and the first flow cell flow path L2 to replace the buffer solution C in the first flow cell flow path L2 and the flow cell C1, until the first flow cell flow path L2, the flow cell C1 and the second flow cell flow path L3 are filled up with the reagent A with a concentration greater than 99%, and the third main flow path L4 contains a mixture of the reagent A and the buffer solution C, as shown in FIG. 7.

Step S2103: The DNA sample fixed inside the flow cell C1 immediately undergoes a biochemical reaction with the reagent A. Concurrently, the second switching component T2 is switched to bring the second main flow path L1 into fluid communication with the bypass flow path L5 while the upstream of the second switching component T2 is disconnected from the flow cell C1; the third switching component T3 is switched to bring the third main flow path L4 into fluid communication with the bypass flow path L5 while the third main flow path L4 downstream of the third switching component T3 is disconnected from the flow cell C1; the first switching component T1 is switched to bring the first main flow path L6 into fluid communication with the second main flow path L1 while the upstream of the first switching component T1 is disconnected from the storage cell C2.

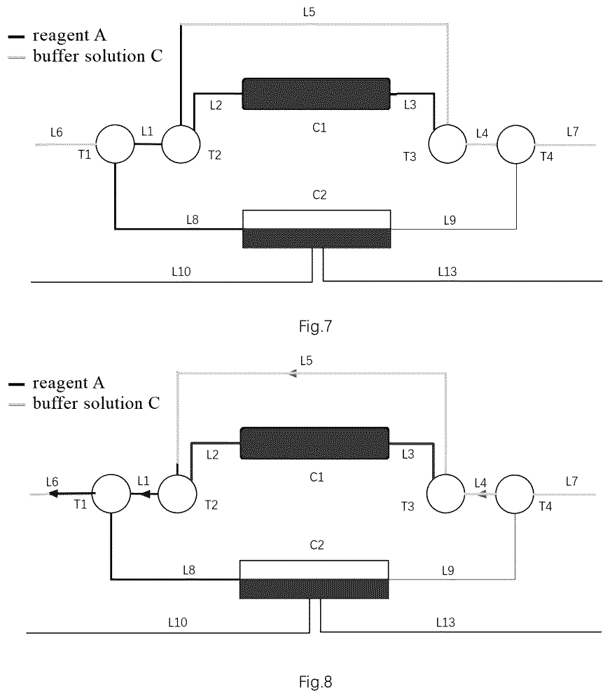

Step S2104: The reagent A in the bypass flow path L5 at an end close to the second switching T2 that meets the concentration requirement is sent back to the second main flow path L1. The reagent A in the second main flow path L1 and the bypass flow path L5, in fluid communication with the first main flow path L6 and downstream of the first switching component T1, enters the first main flow path L6, as shown in FIG. 8. In this step, part of the reagent A is temporarily stored in the first main flow path L6 and then recovered together with part of the reagent in the flow cell C1.

Step S2105: The biochemical reaction in the flow cell C1 is completed. The third switching component T3 is switched to bring the second flow cell flow path L3 into fluid communication with the third main flow path L4, and the fourth switching component T4 is switched to bring the third main flow path L4 into fluid communication with the fourth main flow path L7, thus establishing a fluid communication between the flow cell C1 and the waste-liquid storage area downstream of the fourth main flow path L7; the second switching component T2 is switched to bring the second main flow path L1 into fluid communication with the first flow cell flow path L2, thus establishing a communication between the flow cell C1 and the upstream of the second switching component T2. The buffer solution C with high concentration enters from the first main flow path L6, and passes through the first switching component T1, the second main flow path L1, the second switching component T2 and the first flow cell flow path L2 into the flow cell C1 to replace part of the reagent A recovered from step S2104 in the first main flow path L6, the reagent A in the first flow cell flow path L2, the flow cell C1 and the second flow cell flow path L3, and the mixture of the reagent A and the buffer solution C in the third main flow path L4, and the mixture and the reagent A with low concentration are discharged through the fourth main flow path L7 until the third main flow path L4, the second flow cell flow path L3, and the side of the flow cell C1 close to the third switching component T3 are filled up with the reagent A with high concentration.

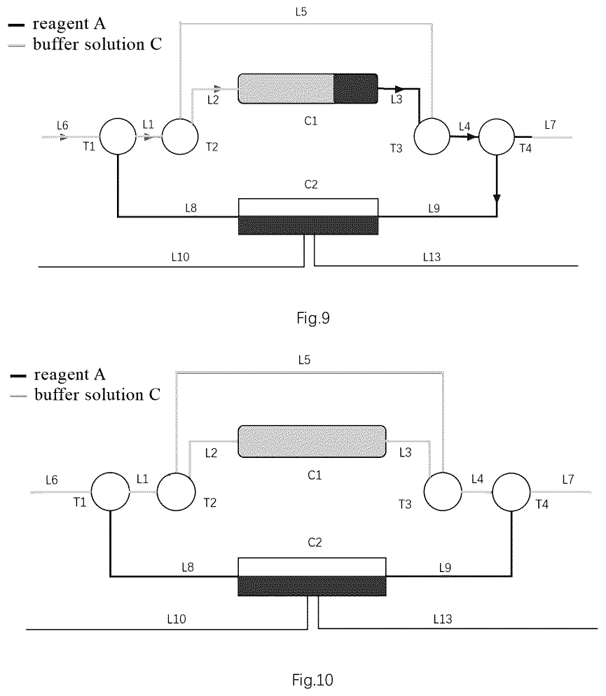

Step S2106: The fourth switching component T4 is switched to establish fluid communication between the third main flow path L4 and the second storage cell connection flow path L9. At this time, the flow cell C1 is in fluid communication with the storage cell C2 through the second flow cell flow path L3, the third switching component T3, the third main flow path L4, the fourth switching component T4 and the second storage cell connection flow path L9. Further, the buffer solution C with high concentration continues to enter the fluid system from the first main flow path L6. With the flow of the buffer solution C, the reagent A with a concentration greater than 95% in the third main flow path L4, the second flow cell flow path L3 and the flow cell C1 enters the second storage cell connection flow path L9 and then enters the storage cell C2, as shown in FIG. 9. So far, the recovery process of the reagent A is completed.

Step S2107: The fourth switching component T4 is switched to establish fluid communication between the third main flow path L4 and the fourth main flow path L7, and at this time, the flow cell C1 is in fluid communication with the waste-liquid storage area downstream of the fourth main flow path L7; subsequently, the reagent A with low concentration and the mixture of the reagent A and the buffer solution C are discharged through the fourth main flow path L7. So far, the first main flow path L6, the second main flow path L1, the first flow cell flow path L2, the bypass flow path L5, the second flow cell flow path L3, the third main flow path L4, the end of the fourth main flow path L7 close to the fourth switching component T4, and the flow cell C1 are all filled up with the buffer solution C.

Step S2108: The first switching component T1 is switched to establish fluid communication between the first main flow path L6 and the second main flow path L1, while the upstream of the first switching component T1 is disconnected from the storage cell C2. The reagent B enters the second main flow path L1 from the first main flow path L6 through the first switching component T1, and then enters the bypass flow path L5 through the second switching component T2 to replace the buffer solution C in the second main flow path L1 and the bypass flow path L5. At this time, the first main flow path L6 and the second main flow path L1 are filled up with the reagent B with a concentration greater than 99%, and the bypass flow path L5 contains a mixture of the reagent B and the buffer solution C. The excess buffer solution C flows sequentially through the third switching component T3, the third main flow path L4, the fourth switching component T4 and the fourth main flow path L7 before being discharged. Step S2108 is to ensure the high concentration of the reagent B entering the flow cell C1.

Step S2109: The state of the first switching component T1 is kept unchanged, so that the first main flow path L6 is still in fluid communication with the second main flow path L1, and the upstream of the first switching component T1 remains disconnected from the storage cell C2; the state of the fourth switching component T4 is kept unchanged, so that the third main flow path L4 is still in fluid communication with the fourth main flow path L7, and the upstream of the fourth switching component T4 remains disconnected from the storage cell C2. The second switching component T2 is switched to bring the second main flow path L1 into fluid communication with the first flow cell flow path L2; and the third switching component T3 is switched to bring the second flow cell flow path L3 into fluid communication with the third main flow path L4, so that both the upstream of the second switching component T2 and the downstream of the third switching component T3 are in fluid communication with the flow cell C1. The reagent B with high concentration enters the flow cell C1 through the second switching component T2 and the first flow cell flow path L2 to replace the buffer solution in the first flow cell flow path L2 and the flow cell C1, until the first flow cell flow path L2, the flow cell C1 and the second flow cell flow path L3 are filled up with the reagent B with a concentration greater than 99%, and the third main flow path L4 contains a mixture of the reagent B and the buffer solution C.

Step S2110: The DNA sample fixed inside the flow cell C1 immediately undergoes a biochemical reaction with the reagent B. Concurrently, the second switching component T2 is switched to bring the second main flow path L1 into fluid communication with the bypass flow path L5 while the upstream of the second switching component T2 is disconnected from the flow cell C1; the third switching component T3 is switched to bring the third main flow path L4 into fluid communication with the bypass flow path L5 while the downstream of the third switching component T3 is disconnected from the flow cell C1.

Step S2111: The buffer solution Centers the second main flow path L1 from the first main flow path L6 through the first switching component T1, and then enters the connected bypass flow path L5 through the second switching component T2 to replace the reagent B with high concentration in the bypass flow path L5 at an end close to the second switching component T2 and that in the second main flow path L1 and the first main flow path L6. The reagent B with low concentration or the mixture of the reagent B and the buffer solution C in the bypass flow path L5 away from the second switching component T2 flows sequentially through the third switching component T3, the third main flow path L4, the fourth switching component T4 and the fourth main flow path L7 before being discharged, until the first main flow path L6, the second main flow path L1 and the bypass flow path L5 are filled up with the buffer solution C with a concentration greater than 99%, and the third main flow path L4 contains a mixture of the reagent B and the buffer solution C.

Step S2112: The biochemical reaction in the flow cell C1 is completed; the third switching component T3 is switched to bring the second flow cell flow path L3 into fluid communication with the third main flow path L4, thus establishing a fluid communication between the flow cell C1 and the waste-liquid storage area downstream of the fourth main flow path L7; the second switching component T2 is switched to bring the second main flow path L1 into fluid communication with the first flow cell flow path L2, thus establishing a communication between the flow cell C1 and the reagent storage chamber upstream of the first main flow path L6. The buffer solution C with high concentration enters the first main flow path L6 and then flows sequentially through the first switching component T1, the second main flow path L1, the second switching component T2 and the first flow cell flow path L2 into the flow cell C1 to replace the reagent B in the first flow cell flow path L2, the flow cell C1 and the second flow cell flow path L3, as well as the mixture of the reagent B and the buffer solution C in the third main flow path L4. The reagent B and the excess buffer solution C in the first flow cell flow path L2, the flow cell C1, the second flow cell flow path L3 and the third main flow path L4 are discharged through the fourth main flow path L7. So far, except for the first storage cell connection flow path L8, the second storage cell connection flow path L9, the storage cell inlet flow path L10, the storage cell outlet flow path L13 and the storage cell C2, all the other flow paths and the flow cell C1 are filled up with the buffer solution C, and the reagent B has been discharged from the fluid system.

Step S2113: Steps S2101~S2112 are cyclically executed for n times, where n is a natural number greater than or equal to 1. For example, n can be 5, 10, 20, or 30. The upper limit of n can be determined based on the reserve of the reagent A in the storage cell C2 and the lower limit of the allowable concentration of the reagent A when participating in molecular biological detection. The more the reserve of the reagent A in the storage cell C2 and the lower the lower limit of the allowable concentration of the reagent A when participating in molecular biological detection, the greater the upper limit of n; conversely, the less the reserve of the reagent A in the storage cell C2 and the higher the lower limit of the allowable concentration of the reagent A when participating in molecular biological detection, the smaller the upper limit of n.

Step S2114: The reagent A in the storage cell C2 is discharged to the waste-liquid storage area through the storage cell outlet flow path L13, and then fresh reagent A enters the storage cell through the storage cell inlet flow path L10 for use in the next round of n times of cycles.

In the logic timing solution of the fluid operation method described above, each time after execution of steps S2101 to S2108, part of the reagent A with a concentration greater than or equal to 95% can be recovered to the storage cell C2.

In the logical timing solution of the fluid operation method described above, the reagent A and the reagent B are separated by the buffer solution C, so they may not come into contact with each other, thereby preventing cross-contamination.

In the logical timing solution of the fluid operation method described above, only two reagents including the reagent A and the reagent B are taken as an example to explain the reagent recovery process, in which the operation performed on the reagent A is to recover part of the reagent A, while the operation performed on the reagent B is not to recover the reagent B. However, according to the above logical timing solution, a corresponding storage flow path may also be provided for the reagent B, and the steps for partial recovery of the reagent A may also be applied to recover the reagent B. Therefore, according to the above method steps, molecular biological detections with the participation of more reagents that need to be partially recovered can be implemented. Additionally, the first main flow path L6 may be selectively communicated with different reagent storage chambers, or the first switching component T1 may be selectively connected to multiple parallel first main flow paths, with each first main flow path connected to a different reagent storage chamber, so as to realize, according to the above method steps, molecular biological detections with the participation of more reagents which do not need to be recovered. Between any two reagents that need to enter the flow cell C1 one after another, the buffer solution C may be used, if necessary, for isolation to avoid cross-contamination caused by mutual contact between the two reagents.

In the logical timing solution of the fluid operation method described above, all the reagents enter the flow cell C1 from the upstream side of the flow cell C1. The reagent A, the reagent B and the buffer solution C may enter the flow cell C1 either by being pushed in using positive pressure from the inlet side of the corresponding flow path upstream of the flow cell C1 or by being sucked in using negative pressure coming from the outlet side of the corresponding flow path downstream of the flow cell C1. For example, if the manner of positive pressure is adopted to push in the reagent A, the reagent A may first be sucked into the first main flow path L6 using negative pressure, and then at the upstream of the first main flow path L6, the reagent A is pushed into the flow cell C1 and the bypass flow path L5 using positive pressure, as shown in FIG. 10.

In the logical timing solution of the fluid operation method described above, the recovery of reagent in the bypass flow path L5 involves sending the reagent back to the upstream of the bypass flow path L5 first, and then recovering this part of the reagent concurrently with the subsequent recovery of reagent in the flow cell C1. This recovery means is suitable for the case where the bypass flow path L5 and the third main flow path L4 have long flow channels. It can prevent the reagent, when recovered through the bypass flow path L5, from being significantly diluted in concentration upon reaching the recovery position through the long flow channel, which could make it difficult to ensure the recovery of the reagent with high concentration. However, if the bypass flow path L5 and the third main flow path L4 have a small internal volume, it is feasible to recover the reagent through the bypass flow path L5.

In the logical timing solution of the fluid operation method described above, the amount of reagent pre-stored in the storage cell C2 plus the amount of the recovered reagent is generally suitable for 20-30 times of cycles, that is, n is 20-30. This is because: 1. every time fresh reagent A enters the storage cell C2, a certain margin should be reserved to avoid pumping in the air when the reagent A is used up; if the number of cycles is small, the frequency of fresh reagent A entering the storage cell C2 becomes high, and the cost of the margin part will be relatively high; 2. after the concentration of the reagent A decreases to a certain extent, it will affect the quality of the reaction in the flow cell C1; if the number of cycles is too large, the concentration of the reagent in the storage cell C2 will decrease, and repeated use of some components in the reagent may have an adverse impact on the quality of the reaction in the flow cell C1.

In the logical timing solution of the fluid operation method described above, the reagent A may be recovered to the storage cell C2. Assuming that the recovered reagent A and the fresh reagent A can be fully mixed in the storage cell C2, if the concentration of the reagent entering the flow cell C1 is required to be greater than 95%, in consideration that the recovered reagent A can be mixed uniformly with the fresh reagent A in the storage cell C2 and the concentration of the reagent A in the storage cell C2 after uniform mixing may be greater than that of the recovered reagent A, the concentration of the recovered reagent A can be less than 95%. Taking into account the pre-stored amount in the storage cell and the cost comprehensively, the recovery ratio can be higher than that in the embodiments shown in FIGS. 2 to 5, and can reach 25% to 35%.

In the logical timing solution of the fluid operation method described above, some steps of the reagent recovery process are performed concurrently with the biochemical reaction occurring in the flow cell C1, which helps save time for molecular biological detection.

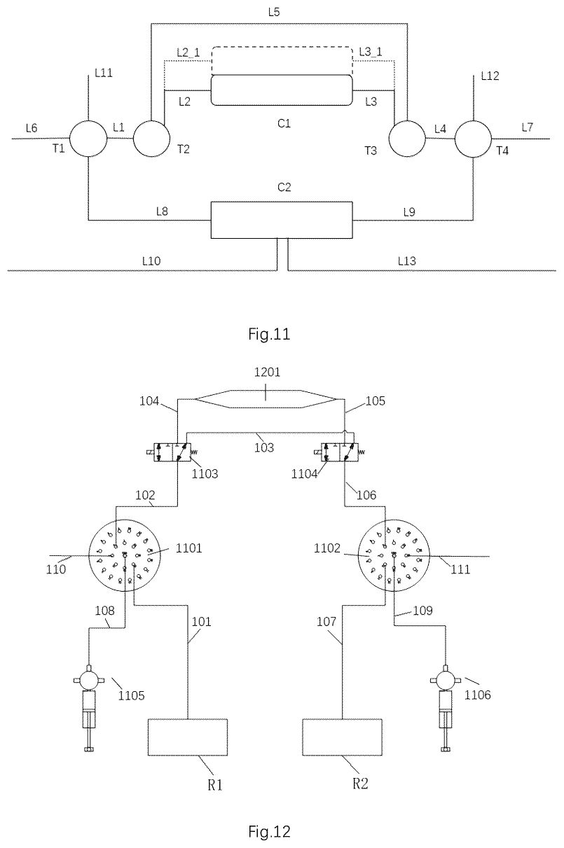

FIG. 11 is a schematic principled view according to an embodiment of the present disclosure.

As shown in FIG. 11, the fluid system in this embodiment of the present disclosure includes a first main flow path L6, a second main flow path L1, a reaction flow path, a bypass flow path L5, a third main flow path L4, a fourth main flow path L7, a first waste-liquid flow path L11 as a branched flow path, a second waste-liquid flow path L12 as a branched flow path, a storage flow path as a branched flow path, a first switching component T1, a second switching component T2, a third switching component T3, and a fourth switching component T4.

The reaction flow path includes a flow cell C1, and a first flow cell flow path L2 and a second flow cell flow path L3 connected to the flow cell C1 respectively. The bypass flow path L5 is connected in parallel with the reaction flow path. The storage flow path includes a storage cell C2, a first storage cell connection flow path L8 and a second storage cell connection flow path L9 connected to the storage cell C2, as well as a storage cell inlet flow path L10 and a storage cell outlet flow path L13 connected to the storage cell. The first waste-liquid flow path L11 is configured to be connected to a waste-liquid storage chamber. The second waste-liquid flow path L12 is configured to be connected to a waste-liquid storage chamber. The first switching component T1 is connected to the first main flow path L6, the second main flow path L1, the first waste-liquid flow path L11 and the first storage cell connection flow path L8, and is configured to selectively communicate any two of the first main flow path L6, the second main flow path L1, the first waste-liquid flow path L11 and the first storage cell connection flow path L8 while having the remaining flow paths disconnected from the two communicated flow paths. The second switching component T2 is connected to the second main flow path L1, the first flow cell flow path L2 and the bypass flow path L5, and is configured to selectively communicate any two of the second main flow path L1, the first flow cell flow path L2 and the bypass flow path L5 while having the remaining flow paths disconnected from the two communicated flow paths. The third switching component T3 is connected to the third main flow path L4, the second flow cell flow path L3 and the bypass flow path L5, and is configured to selectively communicate any two of the third main flow path L4, the second flow cell flow path L3 and the bypass flow path L5 while having the remaining flow paths disconnected from the two communicated flow paths. The fourth switching component T4 is connected to the third main flow path L4, the fourth main flow path L7, the second waste-liquid flow path L12 and the second storage cell connection flow path L9, and is configured to selectively communicate any two of the third main flow path L4, the fourth main flow path L7, the second waste-liquid flow path L12 and the second storage cell connection flow path L9 while having the remaining flow paths disconnected from the two communicated flow paths.

As shown in FIG. 11, in this embodiment, the reaction flow path includes multiple flow cells C1 connected in parallel, so that biochemical reactions can be carried out simultaneously in multiple flow cells C1. Both sides of the multiple flow cells C1 may be connected to the first flow cell flow path L2 and the second flow cell flow path L3 through a first flow cell branch pipe L2_1 and a second flow cell branch pipe L3_1 respectively. The logical timing solution of the feasible fluid operation method in this embodiment of the present disclosure can be made reference to the embodiments shown in FIGS. 2 to 5 and the embodiments shown in FIGS. 6 to 10. The fluid system in this embodiment of the present disclosure has higher flexibility compared with that in the previous embodiments.

Next, an embodiment of the fluid system to which the fluid system of the embodiment of the present disclosure is applied will be described with reference to FIGS. 12 to 20.

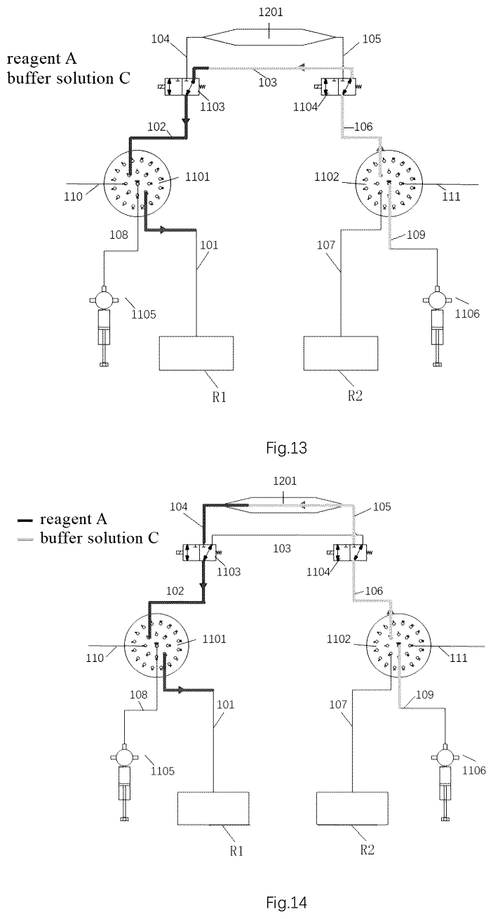

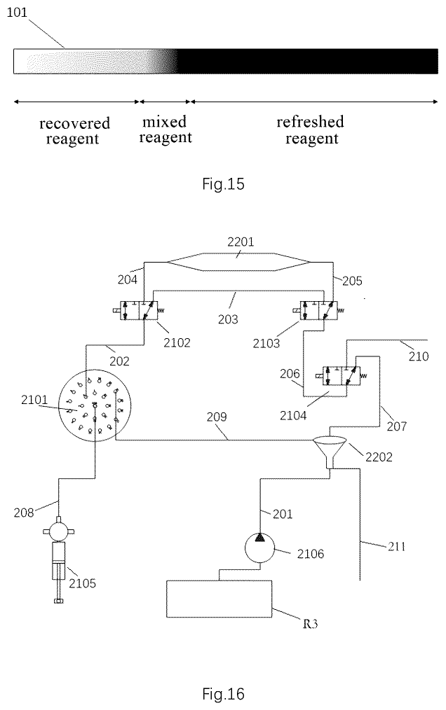

FIGS. 12 to 15 show the principled structure of a fluid system according to an embodiment of the present disclosure. FIG. 12 is a schematic principled structural view of a fluid system according to an embodiment of the present disclosure. FIGS. 13 and 14 are schematic principled structural views of the related fluid operation methods of the fluid system in the embodiment shown in FIG. 12. FIG. 15 is a schematic view showing the distribution of reagents when the recovered reagent, the mixed reagent and a fresh reagent coexist in a conduit in the embodiment shown in FIG. 12, wherein the mixed reagent refers to a mixture of the recovered reagent and the fresh reagent.

As shown in FIGS. 12 to 14, the fluid system in this embodiment is a specific example of the fluid system shown in FIGS. 2 to 5. The first switching component T1 of the fluid system is implemented as a rotary valve 1101. The second switching component T2 of the fluid system is implemented as a solenoid valve 1103. The third switching component T3 of the fluid system is implemented as a solenoid valve 1104. The fourth switching component T4 of the fluid system is implemented as a rotary valve 1102. The first main flow path L6 of the fluid system is implemented as a combination of a conduit 101 and a conduit 108, wherein the conduit 101 is connected to a reagent storage chamber R1, and the conduit 101 is selectively connected with the conduit 108 through the rotary valve 1101. The second main flow path L1 of the fluid system is implemented as a conduit 102; the third main flow path L4 of the fluid system is implemented as a conduit 106; the fourth main flow path L7 of the fluid system is implemented as a combination of a conduit 107 and a conduit 109, wherein the conduit 107 is connected to a reagent storage chamber R2, and the conduit 107 is selectively connected with the conduit 109 through the rotary valve 1102. The bypass flow path L5 of the fluid system is implemented as a conduit 103. The first flow cell flow path L2 of the fluid system is implemented as a conduit 104. The second flow cell flow path L3 of the fluid system is implemented as a conduit 105. The first waste-liquid flow path L11 of the fluid system is implemented as a conduit 110. The second waste-liquid flow path L12 of the fluid system is implemented as a conduit 111. The flow cell C1 is implemented as a chip 1201.

In the embodiments shown in FIGS. 12 to 14, the rotary valve 1101 is connected to a power source 1105 through the conduit 108. The rotary valve 1102 is connected to a power source 1106 through the conduit 109. The conduit 101 is connected to the reagent storage chamber R1, and the conduit 107 is connected to the reagent storage chamber R2. The conduit 110 and the conduit 111 are connected to waste-liquid storage chambers respectively.

In the embodiments shown in FIGS. 12 to 14, the rotary valve 1101 and the rotary valve 1102 are, for example, 25-hole rotary valves; the solenoid valve 1103 and the solenoid valve 1104 are, for example, two-position three-way solenoid valves; the power source 2105 and the power source 1106 are, for example, syringe pumps; and the chip 1201 is, for example, a single-input single-output chip.

The following is a description of the feasible fluid operation method of the fluid system in this embodiment. In this fluid operation method, four basic fluid flow modes are provided, including two normal liquid-passing modes without reagent recovery, namely a bypass liquid-passing mode and a chip liquid-passing mode, and two reagent recovery modes with a reagent recovery process, namely a bypass reagent-recovery mode and a chip reagent-recovery mode. In the feasible logical timing solution of the reagent recovery process, the reagent to be recovered is the reagent A, and the buffer solution C is used to isolate the reagent A. Before recovery of the reagent A, the chip 1201 and the conduits 102 to 106 are filled up with the buffer solution C.

### 1. Bypass Liquid-Passing Mode

Step S3101: the rotary valve 1101 is actuated to communicate the conduit 101 with the conduit 108, and the reagent is pumped into the conduit 108 by the syringe pump 1105 through the conduit 101 and the rotary valve 1101 for temporary storage.

Step S3102: the solenoid valve 1103 is actuated to communicate the conduit 102 with the conduit 103; the solenoid valve 1104 is actuated to communicate the conduit 103 with the conduit 106; and the rotary valve 1101 is actuated to communicate the conduit 108 with the conduit 102, so that the reagent enters the conduit 102 through the conduit 108 and the rotary valve 1101.

Step S3103: the reagent enters the conduit 103 and then enters the conduit 106 through the solenoid valve 1104.

Step S3104: the reagent in the conduit 106 passes through the rotary valve 1102 and is finally discharged through the conduit 111. In this process, no reagent passes through the reaction flow path including the conduit 104, the conduit 105 and the chip 1201.

In this embodiment, the fluid system itself as well as the power sources, the reagent storage chambers and the waste-liquid storage chambers connected to the fluid system are symmetrical in structures. Therefore, reverse pumping can also be carried out using the same logic.

### 2. Chip Liquid-Passing Mode

Step S3201: the rotary valve 1101 is actuated to communicate the conduit 101 with the conduit 108, and the reagent is pumped into 108 by the syringe pump 1105 through the conduit 101 and the rotary valve 1101 for temporary storage.

Step S3202: the solenoid valve 1103 is actuated to communicate the conduit 102 with the conduit 103; the solenoid valve 1104 is actuated to communicate the conduit 103 with the conduit 106; and the rotary valve 1101 is actuated to communicate the conduit 108 with the conduit 102, so that a certain volume of reagent enters the conduit 102 through the conduit 108 and the rotary valve 1101 until the concentration of the reagent at the conduit 102 reaches over 99%, and the reagent with a lower concentration enters the conduit 103 to allow the reagent with higher concentration to enter the chip 1201 later.

Step S3203: the solenoid valve 1103 is actuated to communicate the conduit 102 with the conduit 104, and the solenoid valve 1104 is actuated to communicate the conduit 105 with the conduit 106, so that the reagent enters the conduit 104 and the chip 1201;

Step S3204: after having undergone reaction in the chip 1201, the reagent enters the conduit 106 from the conduit 105 through the solenoid valve 1104;

Step S3205: the reagent in the conduit 106 finally passes through the rotary valve 1102 and the conduit 111 before being discharged.

In this embodiment, the fluid system itself as well as the power sources, the reagent storage chambers and the waste-liquid storage chambers connected to the fluid system are symmetrical in structures. Therefore, reverse pumping can also be carried out using the same logic.

Both of the above two normal liquid-passing modes include the step of pumping the reagent into the conduit 108 for temporary storage, that is, step S3101 and step S3201. For the subsequent steps except for step S3101 and step S3201, the two normal liquid-passing modes can be carried out alternately to meet different detection requirements.

### 3. Bypass Reagent-Recovery Mode

Step S3301: the reagent A enters the chip 1201 from one end of the conduit 101 to complete the corresponding biochemical reaction; the chip 1201, the conduit 102, the conduit 104, the conduit 105, and the side of the conduit 103 close to the conduit 102 contain the reagent A with high concentration; the middle section of the conduit 103 contains a mixture of the reagent A and the buffer solution C; and the side of the conduit 103 close to the conduit 106 as well as the conduit 106 both contain the buffer solution C.

Step S3302: during the reaction in the chip 1201, the rotary valve 1101 is actuated to disconnect the conduit 110 from the conduit 102 and communicate the conduit 101 with the conduit 102; the buffer solution C enters the fluid system through the conduit 107 to recover part of the reagent A in the conduit 103 and the conduit 102 by the bypass fluid-passing mode described above, and this part of the reagent A is recovered into the conduit 101 by the rotary valve 1101 for use in the next reaction.

If the chip reagent-recovery mode follows this step immediately, this step can be stopped after all the reagent A that meets the concentration requirement is delivered to the conduit 102, with no need for sending all the reagent A that meets the recovery conditions into the conduit 101, as shown in FIG. 13.

### 4. Chip Reagent-Recovery Mode

The chip reagent-recovery mode is generally carried out immediately after the bypass reagent-recovery mode. After the reaction in the chip 1201 is completed, the buffer solution Centers the fluid system from the conduit 101 by the chip liquid-passing mode to recover part of the reagent A in the conduit 104, the conduit 105 and the chip 1201.

If the conduit 102 has undergone other pumping processes during the biochemical reaction in the chip 1201, the rotary valve 1101 needs to be actuated to communicate the conduit 102 with the conduit 110 so as to discharge the reagent with low concentration or other reagents in the conduit 102 through the conduit 110, before execution of the chip reagent-recovery mode, and then, the rotary valve 1101 is actuated to communicate the conduit 102 with the conduit 101 to recover the reagent A that meets the concentration requirement into the conduit 101 through the rotary valve 1101 for use in the next reaction, as shown in FIG. 14.

In the two reagent recovery modes described above, if the reagent A enters the fluid system from the conduit 101, the buffer solution C can enter the fluid system from the conduit 107. In the conduit 101, the reagent at an end close to the rotary valve 1101 may be refreshed, and part of the reagent at an end close to the reagent storage chamber R1 may be sent back to the reagent storage chamber R1 so as to ensure that the volume of the reagent in the conduit 101 is greater than that of the recovered part of the reagent, as shown in FIG. 15, to ensure that the reagent sent back to the reagent storage chamber R1 cannot come into contact with the recovered reagent.

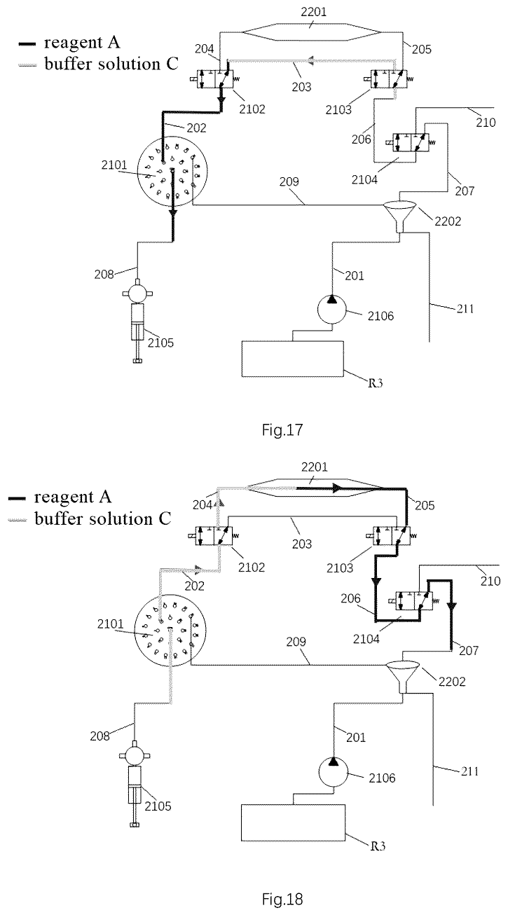

FIGS. 16 to 18 show the principled structure of a fluid system according to an embodiment of the present disclosure. FIG. 16 is a schematic principled structural view of a fluid system according to an embodiment of the present disclosure. FIGS. 17 and 18 are schematic principled structural views of the related fluid operation methods of the fluid system in the embodiment shown in FIG. 16.

As shown in FIGS. 16 to 18, the fluid system in this embodiment is a specific example of the fluid system shown in FIGS. 6 to 10. The first switching component T1 of the fluid system is implemented as a rotary valve 2101. The second switching component T2 of the fluid system is implemented as a solenoid valve 2102. The third switching component T3 of the fluid system is implemented as a solenoid valve 2103. The fourth switching component T4 of the fluid system is implemented as a solenoid valve 2104. The first main flow path L6 of the fluid system is implemented as a conduit 208; the second main flow path L1 of the fluid system is implemented as a conduit 202; the third main flow path L4 of the fluid system is implemented as a conduit 206; the fourth main flow path L7 of the fluid system is implemented as a conduit 210. The bypass flow path L5 of the fluid system is implemented as a conduit 203. The first flow cell flow path L2 of the fluid system is implemented as a conduit 204.

The second flow cell flow path L3 of the fluid system is implemented as a conduit 205. The first storage cell connection flow path L8 of the fluid system is implemented as a conduit 209. The second storage cell connection flow path L9 of the fluid system is implemented as a conduit 207. The flow cell C1 is implemented as a chip 2201. The storage cell C2 is implemented as a storage cell 2202. The storage cell inlet flow path L10 is implemented as a conduit 201. The storage cell outlet flow path L13 is implemented as a conduit 211.

In the embodiments shown in FIGS. 16 to 18, the conduit 208 is connected to a power source 2105. The conduit 201 is connected to a reagent storage chamber R3 through a liquid pump 2106.

In the embodiments shown in FIGS. 16 to 18, the rotary valve 2101 is, for example, a 25-hole rotary valve; the solenoid valve 2102, the solenoid valve 2103 and the solenoid valve 2104 are, for example, two-position three-way solenoid valves; the power source 1105 is, for example, a syringe pump; the liquid pump 2106 is, for example, a diaphragm liquid pump; the chip 2201 is, for example, a single-input single-output chip; the storage cell 2202 is a measuring cup with an open top, with the bottom of the measuring cup being provided with an opening connected with the conduit 201.

The following is a description of the feasible fluid operation method of the fluid system in this embodiment. In this fluid operation method, three basic fluid flow modes are provided, including two normal liquid-passing modes without reagent recovery, namely a bypass liquid-passing mode and a chip liquid-passing mode, and one reagent recovery mode with a reagent recovery process, namely a chip reagent-recovery mode. In the feasible logical timing solution of the reagent recovery process, the reagent to be recovered is the reagent A, and the buffer solution C is used to isolate the reagent A. Before recovery of the reagent A, the chip 2201 and the conduits 202 to 206 are filled up with the buffer solution C.

The reagent in the storage cell 2202 may be refreshed as required. For example, in this embodiment, every time after the reagent in the storage cell 2202 has participated in 20 times of cycles, the reagent in the storage cell 2202 will be discharged through the conduit 211, and new reagent will be pumped into the storage cell 2202 from the reagent storage chamber R3 by the liquid pump 2106 to ensure freshness of the reagent in the storage cell 2202.

### 1. Bypass Liquid-Passing Mode

Step S4101: the rotary valve 2101 is actuated to communicate the conduit 209 with the conduit 208, and the reagent is pumped into the storage cell 2202 through the liquid pump 2106 and the conduit 201, and then pumped into the conduit 208 by the power source 2105 through the conduit 209 and the rotary valve 2101 for temporary storage;

Step S4102: the solenoid valve 2102 is actuated to communicate the conduit 202 with the conduit 203; the solenoid valve 2103 is actuated to communicate the conduit 203 with the conduit 206; the solenoid valve 2104 is actuated to communicate the conduit 206 with the conduit 210; and the rotary valve 2101 is actuated to communicate the conduit 202 with the conduit 208 so that the reagent enters the conduit 202 through the conduit 208 and the rotary valve 2101;

Step S4103: the reagent enters the conduit 203 and then enters the conduit 206 through the solenoid valve 2103;

Step S4104: the reagent in the conduit 206 passes through the solenoid valve 2104 and is finally discharged from the conduit 210.

In the bypass liquid-passing mode, no reagent passes through the reaction flow path including the conduit 204, the conduit 205 and the chip 2201.

### 2. Chip Liquid-Passing Mode

Step S4201: the rotary valve 2101 is actuated to communicate the conduit 209 with the conduit 208, and the reagent is pumped into the storage cell 2202 through the liquid pump 2106 and the conduit 201, and then pumped into the conduit 208 by the power source 2105 through the conduit 209 and the rotary valve 2101 for temporary storage;

Step S4202: the solenoid valve 2102 is actuated to communicate the conduit 202 with the conduit 203; the solenoid valve 2103 is actuated to communicate the conduit 203 with the conduit 206; the solenoid valve 2104 is actuated to communicate the conduit 206 with the conduit 210; and the rotary valve 2101 is actuated to communicate the conduit 202 with the conduit 208, so that the reagent enters the conduit 202 through the conduit 208 and the rotary valve 2101 to make the concentration of the reagent at the conduit 202 reach over 99%, and the reagent with lower concentration enters the conduit 203 so as to allow the reagent with higher concentration to enter the chip 2201 later;

Step S4203: the solenoid valve 2102 is actuated to communicate the conduit 202 with the conduit 204, and the solenoid valve 2103 is actuated to communicate the conduit 205 with the conduit 206, so that the reagent enters the conduit 204 and the chip 2201;

Step S4204: after having undergone reaction in the chip 2201, the reagent enters the conduit 206 from the conduit 205;

Step S4205: the reagent in the conduit 206 is discharged after passing through the solenoid valve 2104 and the conduit 210.

In the chip liquid-passing mode, no reagent may pass through the conduit 203 as the bypass flow path L5.

Both of the above two normal liquid-passing modes include the step of pumping the reagent into the conduit 208 for temporary storage, that is, step S4101 and step S4201. For the subsequent steps except for step S4101 and step S4201, the two normal liquid-passing modes can be carried out alternately to meet different detection requirements.

### 3. Chip Reagent-Recovery Mode

In this embodiment, the conduit 203, serving as the bypass flow path L5, has a certain length. The recovery of the reagent in the conduit 203 is carried out relying on the chip 2201. Therefore, only one reagent recovery mode, namely the chip reagent-recovery mode, is provided.

Step S4301: the reagent A enters the chip 2201 by the chip liquid-passing mode to complete the biochemical reaction. At the beginning of the biochemical reaction, the chip 2201, the conduit 202, the conduit 204, the conduit 205, and the side of the conduit 203 close to the conduit 202 all contain the reagent A with higher concentration; at the middle section of the conduit 203 contains a mixture of the reagent A and the buffer solution C; and the side of the conduit 203 close to the conduit 206 as well as the conduit 206 both contain the buffer solution C. During the reaction in the chip 2201, the reagent A in the conduit 203 that meets the concentration requirements is recovered by negative pressure: the solenoid valve 2102 is actuated to communicate the conduit 202 with the conduit 203; the solenoid valve 2103 is actuated to communicate the conduit 203 with the conduit 206; the solenoid valve 2104 is actuated to communicate the conduit 206 with the conduit 210; and the rotary valve 2101 is actuated to communicate the conduit 202 with the conduit 208; the reagent A whose concentration reaches the standard is pumped back to the conduit 202 by the power source 2105, and if the volume of the reagent A is large enough, the reagent A may also enter the rotary valve 2101 and the conduit 208.

Step S4302: after the reaction in the chip 2201 is completed, the solenoid valve 2102 is actuated to communicate the conduit 202 with the conduit 204; the solenoid valve 2103 is actuated to communicate the conduit 205 with the conduit 206; and the power source 2105 pushes liquid and sends part of the reagent A recovered in the previous step back into the conduit 202. Then, the buffer solution Centers from another hole of the rotary valve 2101, and flows by the chip liquid-passing mode to firstly discharge the reagent A with lower concentration and the buffer solution C in the conduit 205 and the conduit 206 from the conduit 210; then, the solenoid valve 2104 disconnects the conduit 206 from the conduit 210 and instead communicates the conduit 206 with the conduit 207; and the buffer solution C further enters the chip 2201 to send the reagent A that meets the concentration requirements in the conduit 202, the conduit 204, the chip 2201, the conduit 205 and the conduit 206 into the conduit 207 and the storage cell 2202 through the solenoid valve 2104 for use in the next reaction.

If there are other reagents, besides the buffer solution C, passing through the conduit 203 during the biochemical reaction in the chip 2201, step S4301 will not be carried out, and step S4302 may be carried out directly, namely, not recovering the reagents in the conduit 203 and the conduit 202, and only recovering the reagents in the reaction flow path.

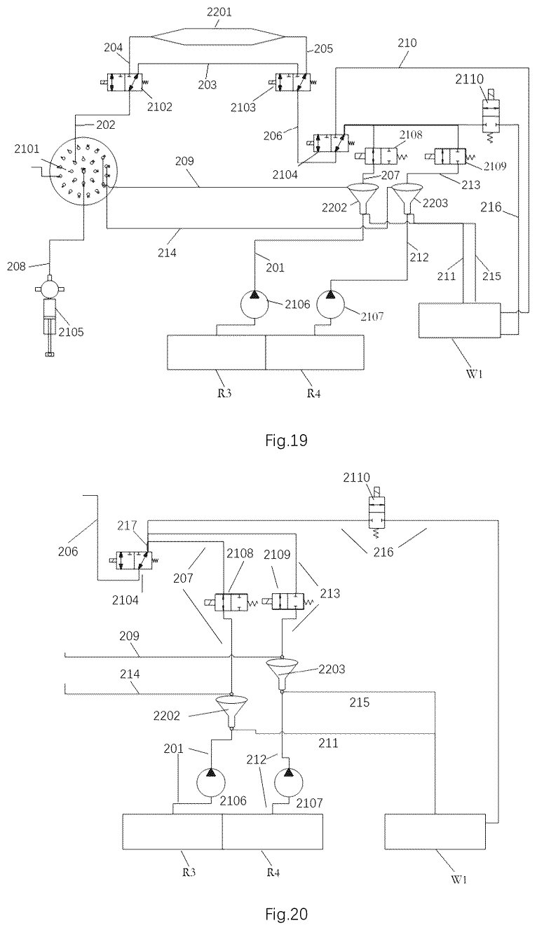

FIGS. 19 to 20 show the principled structure of a fluid system according to an embodiment of the present disclosure. FIG. 19 is a schematic principled structural view of a fluid system according to an embodiment of the present disclosure. FIG. 20 is a partial schematic principled structural view of the fluid system of FIG. 19, wherein FIG. 20 mainly shows the differences between the fluid system in this embodiment and the fluid system shown in FIGS. 16 to 18.

As shown in FIGS. 19 to 20, the fluid system in this embodiment is added with one storage flow path on the basis of the fluid system in the embodiment shown in FIGS. 16 to 18. Only the differences between this embodiment and the embodiment shown in FIGS. 16 to 18 will be described below, and the parts not described in this embodiment can all be made reference to the embodiments shown in FIGS. 16 to 18.

As shown in FIGS. 19 to 20, compared with the embodiment shown in FIGS. 16 to 18, the fluid system further includes a storage cell 2203 that is connected in parallel with the storage cell 2202. The storage cell 2203 is connected with the rotary valve 2101 through a conduit 214, is connected with the solenoid valve 2104 through a conduit 213, is connected with a reagent storage chamber R4 through a conduit 212 that is equipped with a liquid pump 2107 such as a diaphragm liquid pump, and is connected with the waste-liquid storage chamber W1 through a conduit 215. The conduit 211 and the conduit 210 are also connected with the waste-liquid storage chamber W1. The conduit 216 connects the solenoid valve 2104 with the waste-liquid storage chamber W1. A solenoid valve 2108 is provided on the conduit 207 to control the on-off of the conduit 207. A solenoid valve 2109 is provided on the conduit 213 to control the on-off of the conduit 213. A solenoid valve 2110 is provided on the conduit 216 to control the on-off of the conduit 216. The solenoid valve 2108, the solenoid valve 2109 and the solenoid valve 2110 are, for example, two-position two-way solenoid valves. The conduit 207, the conduit 213 and the conduit 216 are all connected with the solenoid valve 2104 through the conduit 217.

The storage cell 2203 is a measuring cup with an open top, with the bottom of the measuring cup being provided with an opening connected with the conduit 212. Similar to the storage cell 2202, the reagent in the storage cell 2203 may be refreshed as required. For example, in this embodiment, every time after the reagent in the storage cell 2203 participates in 20 times of cycles, the reagent in the storage cell 2203 may be discharged from the conduit 215 to the waste-liquid storage chamber W1, and a new reagent will be pumped into the storage cell 2203 from the reagent storage chamber R4 by the liquid pump 2107 to ensure the freshness of the reagent in the storage cell 2203.

The following is a description of the feasible fluid operation method of the fluid system in this embodiment. In this fluid operation method, three basic fluid flow modes are provided, including two normal liquid-passing modes without reagent recovery, namely a bypass liquid-passing mode and a chip liquid-passing mode, and one reagent recovery mode with a reagent recovery process, namely a chip reagent-recovery mode. In a feasible logical timing solution of the reagent recovery process, the reagent to be recovered is the reagent A and the reagent B, and the buffer solution C is used to isolate the reagent A from the reagent B.

Before recovery of the reagent A and the reagent B, the chip 2201 and the conduits 202 to 206 are filled up with the buffer solution C. The reagent A is stored in the reagent storage chamber R3 and the storage cell 2202, and the reagent B is stored in the reagent storage chamber R4 and the storage cell 2203.

For the bypass liquid-passing mode and the chip liquid-passing mode, refer to the relevant explanation in the embodiment shown in FIGS. 16 to 18.

The chip reagent-recovery mode is as following:

S5301: the reagents to be recovered enter the corresponding storage cells through the solenoid valve 2104. Assuming that the reagent A is to be recovered first, it can be recovered according to the chip reagent-recovery mode in the embodiment shown in FIGS. 16 to 18. In the recovery process, the solenoid valve 2108 is opened, the solenoid valve 2109 and the solenoid valve 2110 are closed, and the reagent A is recovered into the storage cell 2202 through the conduit 217 and the conduit 207.

S5302: after recovery of the reagent A, there may be the reagent A remaining in the conduit 217. Therefore, if the reagent B needs to be recovered, it is necessary to take away this part of the reagent A using the buffer solution C first. At this time, the solenoid valve 2108 and the solenoid valve 2109 are closed, the solenoid valve 2110 is opened, and the buffer solution C flows to the conduit 216 through the solenoid valve 2104 and the conduit 217. In this step, it needs to ensure that the conduit 217 is filled up with the buffer solution C.

S5303: the solenoid valve 2108 and the solenoid valve 2019 are kept closed, and the reagent B enters the conduit 216 from the solenoid valve 2104 through the conduit 217. In this step, it needs to ensure that the reagent B with a concentration of more than 99% fills up the conduit 217.

S5304: the solenoid valve 2109 is opened and the solenoid valve 2110 is closed. The reagent B is recovered into the storage cell 2203 through the conduit 217 and the conduit 213. Up to this point, the recovery of the two reagents is completed.

In this embodiment, the conduit 217 should be made as short as possible to recover as much reagent as possible. Of course, assuming that the reagent A and the reagent B are not allowed to contact each other at all, even if the conduit 217 is very short, it needs to introduce the buffer solution C for isolation during the reagent recovery process. If the reagent A and the reagent B can be in slight contact with each other, the buffer solution C is not needed for isolation.

In this embodiment, each reagent has an independent storage cell. If more reagents are to be recovered, more branches may be added to the conduit 217 for connection with more storage flow paths, and valves may be adopted to ensure that the reagents can flow to the corresponding storage flow paths independently. The valves for switching the storage flow paths are not limited to the two-position two-way solenoid valves provided on each branch in this embodiment. For example, rotary valves may also be used to realize the switching of the multiple storage flow paths.

In the embodiments of the present disclosure, the descriptions related to concentration, for example, ensuring that the second main flow path L1 contains the reagent A with a concentration of 99%, etc., all concern conversions relative to the concentration of the reagent entering the system in the current round. For example, when the reagent A enters the entire fluid system for the first time, the initial concentration is 1, and the concentration is required to be more than 99%. After recovery, the reagent A entering the flow cell C1 for the second time has been premixed with the recovery part, the initial concentration has already been less than 1, such as 0.98, and the 99% at this time refers to 99% of 0.98, that is, 0.97. In the entire reagent-recovery process, it is ensured that the amount of reagent entering the flow cell in each round is fixed, and the concentration of the reagent entering the flow cell may be slightly changed. The concentration of the reagent entering the flow cell shall be within a reasonable range. For example, in each embodiment of the present disclosure, it is required that the stable concentration of the reagent after entering the flow cell C1 is more than 95% of the initial concentration.

On the premise of recovering reagents with a concentration of over 95%, about 25% of the reagent can be recovered at most each time in the embodiment shown in FIGS. 2 to 5, and about 35% of the reagent can be recovered at most each time in the embodiment shown in FIGS. 6 to 10. As the requirement for the concentration of the recovered reagent is very high, the recovery ratio is already quite high.

Based on the above description, the embodiments of the present disclosure have at least one of the following advantages:

Before the reagent enters the chip, the part with low concentration can be sent to the bypass flow path first, which ensures the concentration of the reagent entering the chip and can also help reduce cross-contamination.

Due to the use of the buffer solution to isolate the reagents, the recovered reagent is slightly diluted by the buffer solution, but there is no mixing with other reagents, thus effectively avoiding cross-contamination of the reagents.

The process of discharging the reagent with low concentration and the process of recovering part of the reagent with high concentration in the reagent recovery process can be carried out concurrently with the biochemical reaction within the flow cell or be completed in the process of the next reagent entering the flow cell. This reduces the waiting time and does not affect the total process time of detection.

Use of a single agent and simultaneous use of multiple reagents are both allowed. In case of the simultaneous use of multiple reagents, any one or several of the reagents may be selected for recovery.

The recovered reagent whose concentration reaches the standard can enter the part connected with the reagent storage chamber, and the recovered reagent can avoid entering the reagent storage chamber. Alternatively, it can enter a relatively independent storage cell, rather than being sent back to the flow cell after passing through the flow cell. Therefore, the embodiments of the present disclosure are beneficial to reducing cross-contamination.

In the reagent recovery process, part of the reagent recovery is carried out concurrently with the biochemical reaction, and the other part of reagent recovery is carried out when the buffer solution enters the flow cell C1 in the next step. Therefore, the recovery of the reagent does not affect the working efficiency of the molecular biological detection device.

A multi-channel chip with multiple of the flow cells in parallel and with multiple inputs and multiple outputs can be applied to the fluid system in the embodiments of the present disclosure.

The driving mechanism for driving the fluid to flow can be flexibly selected. For example, the flow of the fluid in the fluid system can be driven by positive pressure or negative pressure. Different reagents can be recovered in one direction or in multiple directions. The driving mechanism can be a mechanical pump, such as a diaphragm pump or a syringe pump. In addition, other power systems such as pneumatic, electroosmotic microfluidic and digital microfluidic systems can also achieve the same effect.

When the fluid system according to the embodiments of the present disclosure is applied, it is not limited by the form of the flow cell such as the size and the number of flow channels.

Finally, it should be explained that the above embodiments are only used to illustrate but not to limit the technical solution of the embodiments in the present disclosure. Although the embodiments of the present disclosure have been described in detail with reference to the preferred embodiments, those skilled in the art should understand that: they can also modify the specific implementing modes of the embodiments of the present disclosure or make equivalent replacement as for some technical features; and these modifications or replacement without departing from the spirit of the technical solution of the embodiments of the present disclosure should be included in the scope of the technical solution claimed by the present disclosure.

1. A fluid system, comprising:
one or more first main flow paths configured to be connected with at least one reagent storage chamber;
a second main flow path;
a reaction flow path comprising a flow cell;
a bypass flow path connected in parallel with the reaction flow path;
a third main flow path;
one or more branched flow paths; and
a plurality of switching components, the plurality of switching components comprising:
a first switching component, which is connected to the one or more first main flow paths, the second main flow path and at least one of the one or more branched flow paths, and is configured such that the second main flow path is selectively communicated with any one of the one or more first main flow paths and the at least one of the one or more branched flow paths, while the remaining flow paths connected with the first switching component are disconnected;
a second switching component, which is connected to the second main flow path, the reaction flow path and the bypass flow path, and is configured such that the second main flow path is selectively communicated with either one of the reaction flow path and the bypass flow path, while the remaining flow paths connected with the second switching component are disconnected; and
a third switching component, which is connected to the third main flow path, the reaction flow path and the bypass flow path, and is configured such that the third main flow path is selectively communicated with either one of the reaction flow path and the bypass flow path, while the remaining flow paths connected with the third switching component are disconnected.

2. The fluid system according to claim 1, wherein the reaction flow path further comprises:
a first flow cell flow path connecting the flow cell with the second switching component; or
a second flow cell flow path connecting the flow cell with the third switching component; or
a first flow cell flow path connecting the flow cell with the second switching component and a second flow cell flow path connecting the flow cell with the third switching component.

3. The fluid system according to claim 1, wherein the reaction flow path comprises two or more flow cells arranged in parallel.

4. The fluid system according to claim 1, wherein the one or more branched flow paths comprise a first waste-liquid flow path, which is connected with the first switching component and configured to be connected with a waste-liquid storage chamber.

5. The fluid system according to claim 1, wherein
the fluid system further comprises one or more fourth main flow paths configured to be connected with at least one reagent storage chamber, or with at least one waste-liquid storage chamber, or with at least one reagent storage chamber and at least one waste-liquid storage chamber;
the plurality of switching components further comprise a fourth switching component, which is connected to the third main flow path, the one or more fourth main flow paths and at least one of the one or more branched flow paths, and configured such that the third main flow path is selectively communicated with any one of the one or more fourth main flow paths and the at least one of the one or more branched flow paths, while the remaining flow paths connected with the fourth switching component are disconnected.

6. The fluid system according to claim 5, wherein the one or more branched flow paths comprise a second waste-liquid flow path, which is connected with the fourth switching component and configured to be connected with a waste-liquid storage chamber.

7. The fluid system according to claim 5, wherein the one or more branched flow paths comprise a storage flow path, which comprises:
a storage cell;
a first storage cell connection flow path connecting the first switching component with the storage cell; and
a second storage cell connection flow path connecting the fourth switching component with the storage cell.

8. The fluid system according to claim 7, wherein the storage flow path further comprises at least one of a storage cell inlet flow path connected with the storage cell a storage cell outlet flow path connected with the storage cell.

9. The fluid system according to claim 7, wherein the one or more branched flow paths comprise a plurality of the storage flow paths arranged in parallel.

10-11. (canceled)

12. A biochemical analysis and detection platform, comprising the fluid system according to claim 1.

13-14. (canceled)

15. A fluid operation method of the fluid system according to claim 1, wherein the fluid operation method comprises:
allowing a reagent to enter at least an end of the bypass flow path close to the second switching component as well as the reaction flow path through the second main flow path;
disconnecting the reaction flow path from the second main flow path and the third main flow path, the reagent being undergoing biochemical reactions within the flow cell of the reaction flow path; and
recovering the reagent in at least one of the bypass flow path or the reaction flow path.

16. The fluid operation method according to claim 15, wherein while the reagent is undergoing biochemical reactions within the flow cell of the reaction flow path, the reagent in the bypass flow path is recovered.

17. The fluid operation method according to claim 16, wherein part of the reagent in the bypass flow path is made to flow to the second main flow path so as to recover the reagent in the bypass flow path through the second main flow path.

18. The fluid operation method according to claim 15, wherein the reagent in the reaction flow path is made to flow to the second main flow path so as to recover the reagent A in the reaction flow path through the second main flow path.

19. The fluid operation method according to claim 18, wherein the fluid operation method further comprises allowing the reagent A, recovered through the second main flow path, to flow to the first main flow path.

20. The fluid operation method according to claim 18, wherein the fluid operation method further comprises allowing the reagents, recovered from the bypass flow path and the reaction flow path through the second main flow path, to flow to the third main flow path to recover the reagent A through the third main flow path.

21. The fluid operation method according to claim 20, wherein the fluid system further comprises a storage flow path connected with the first switching component and a fourth switching component; and the fluid operation method comprises allowing the reagent, which is recovered through the third main flow path, to flow to the storage flow path.

22. The fluid operation method according to claim 15, wherein the fluid operation method comprises pushing the reagent to flow within the fluid system by a buffer solution to recover the reagent.

23. The fluid operation method according to claim 22, wherein the fluid operation method comprises discharging the buffer solution or a mixture of the buffer solution and the reagent whose concentration is less than a predetermined concentration into a waste-liquid storage chamber.

24. The fluid operation method according to claim 23, wherein
the fluid operation method comprises discharging the buffer solution or the mixture of the buffer solution and the reagent whose concentration is less than a predetermined concentration to the waste-liquid storage chamber through at least one of the one or more branched flow paths;
the fluid system comprises a fourth main flow path, which is selectively communicated with the third main flow path and configured to be connected with the waste-liquid storage chamber; and the fluid operation method comprises discharging the buffer solution or the mixture of the buffer solution and the reagent whose concentration is less than a predetermined concentration to the waste-liquid storage chamber through the fourth main flow path; or
the fluid operation method comprises discharging the buffer solution or the mixture of the buffer solution and the reagent whose concentration is less than a predetermined concentration to the waste-liquid storage chamber through at least one of the one or more branched flow paths, and the fluid system comprises a fourth main flow path, which is selectively communicated with the third main flow path and configured to be connected with the waste-liquid storage chamber; and the fluid operation method comprises discharging the buffer solution or the mixture of the buffer solution and the reagent whose concentration is less than a predetermined concentration to the waste-liquid storage chamber through the fourth main flow path.
